SEQUENCE LISTING (22 sequences)

SEQ ID NO: 1 - protein, 113 aa, Mus musculus
DIVITQSTKFMSTSVGDRVSITCKASQDVSTAVAWYQQKPGQSPKLLIYWASTRHTGVPDRFTGSGSGTDYTLTISRVQAEDLALYYCQQHYTTPWTFGGGTKLEIKRTVAAP

SEQ ID NO: 2 - protein, 112 aa, Mus musculus
EVKLEESGGGLVQPGGSRKLSCAASGFTFSSFGMHWVRQAPEKGLEWVAYISSGSSTLHYADTVKGRFTISRDNPKNTLFLQMKLPSLCYGLLGSRNLSHRLLSQNDTPICL

SEQ ID NO: 3 - protein, 214 aa, Artificial Sequence
  Chimeric light chain
DIVITQSTKFMSTSVGDRVSITCKASQDVSTAVAWYQQKPGQSPKLLIYWASTRHTGVPDRFTGSGSGTDYTLTISRVQAEDLALYYCQQHYTTPWTFGGGTKLEIKRTVAAPSFVIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, Artificial Sequence
  Chimeric heavy chain
EVKLEESGGGLVQPGGSRKLSCAASGFTFSSFGMHWVRQAPEKGLEWVAYISSGSSTLHYADTVKGRFTISRDNPKNTLFLQMKLPSLCYGLLGSRNLSHRLLSQNDTPICLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 230 aa, Artificial Sequence
  Chimeric light chain
QLLSCIALSLALVTNSDIVITQSTKFMSTSVGDRVSITCKASQDVSTAVAWYQQKPGQSPKLLIYWASTRHTGVPDRFTGSGSGTDYTLTISRVQAEDLALYYCQQHYTTPWTFGGGTKLEIKRTVAAPSFVIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 4175 bp, Artificial Sequence
  Chimeric light chain
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagacatagttattacccaaagtacaaaattcatgtctacatccgtgggtgaccgggtttcaatcacctgtaaggccagccaggatgtgtcaacagcagttgcttggtaccagcagaagcctggacagagccctaagctcctgatatattgggcttccacccggcatacaggtgtcccagataggttcacaggctccggaagtggtactgactacacactgactatcagccgcgtgcaggcagaagacctcgctctgtattattgtcagcagcattatacaactccctggaccttcggaggaggaacaaagctggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 7 - DNA, 4768 bp, Artificial Sequence
  Chimeric heavy chain
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggtgaattcactatggaagttaagctggaagaatctggtggtgggctcgttcaacccggaggaagcaggaagctctcctgcgctgcatcaggctttaccttctccagtttcggaatgcattgggttaggcaggctcccgagaagggactggagtgggtggcatacattagtagcggcagtagcaccctgcattacgcagacacagtcaagggccggttcacaatatcacgggataaccctaagaacacactgtttctgcagatgaagctgccctctctctgttacggcctcctgggcagccggaatctgtcacatagactgctctcccagaacgatacacccatttgcctgacagttagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 8 - DNA, 37 bp, Artificial Sequence
  Forward primer
atatcatatgtacaacctggacgtgcggggcgcgcgg

SEQ ID NO: 9 - DNA, 40 bp, Artificial Sequence
  Reverse primer
atatgcggccgccatctgcttctcatacaccacgtcaacc

SEQ ID NO: 10 - DNA, 37 bp, Artificial Sequence
  Forward primer
atatcatatgtcccggcccgtggtggatatggtcacc

SEQ ID NO: 11 - protein, 961 aa, Homo sapiens
FGYRVLQVGNGVIVGAPGEGNSTGSLYQCQSGTGHCLPVTLRGSNYTSKYLGMTLATDPTDGSILFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQM

SEQ ID NO: 12 - protein, 476 aa, Homo sapiens
RPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQM

SEQ ID NO: 13 - protein, 5 aa, Artificial Sequence
  Complementary Determining Region
SFGMH

SEQ ID NO: 14 - protein, 17 aa, Artificial Sequence
  Complementary Determining Region
YISSGSSTLHYADTVKG

SEQ ID NO: 15 - protein, 11 aa, Artificial Sequence
  Complementary Determining Region
GSRNLSHRLLS

SEQ ID NO: 16 - protein, 11 aa, Artificial Sequence
  Complementary Determining Region
KASQDVSTAVA

SEQ ID NO: 17 - protein, 7 aa, Artificial Sequence
  Complementary Determining Region
WASTRHT

SEQ ID NO: 18 - protein, 9 aa, Artificial Sequence
  Complementary Determining Region
QQHYTTPWT

SEQ ID NO: 19 - protein, 108 aa, Artificial Sequence
  Chimeric light chain variable domain
DIVMTQSPSLLSASVGDRVTITCKASQDVSTAVAWYQQKPGKAPKLLIYWASTRHTGVPSRFSGSGSGTDFTLTISSLQPEDFALYYCQQHYTTPWTFGGGTKVEIKR

SEQ ID NO: 20 - protein, 108 aa, Artificial Sequence
  Chimeric light chain variable domain
DILMTQSPSLLSASVGDRVTITCKASQDVSTAVAWYQQKPGKAPKLLIYWASTRHTGVPSRFTGSGSGTDFTLTISRLQAEDFALYYCQQHYTTPWTFGGGTKVEIKR

SEQ ID NO: 21 - protein, 121 aa, Artificial Sequence
  Chimeric heavy chain variable domain
EVQLVESGGGLVQPGGSLRLSCAASGFTFSSFGMHWVRQAPGKGLEWVSYISSGSSTLHYADTVKGRFTISRDNSKNSLYLQMNSLRAEDTAVYYCARGSRNLSHRLLSYWGQGTLVTVSS

SEQ ID NO: 22 - protein, 121 aa, Artificial Sequence
  Chimeric heavy chain variable domain
EVKLVESGGGLVQPGGSLRLSCAASGFTFSSFGMHWVRQAPEKGLEWVAYISSGSSTLHYADTVKGRFTISRDNSKNSLYLQMNLLRAEDTAVYYCARGSRNLSHRLLSYWGQGTLVTVSS

(19) United States
(12) Patent Application Publication
(10) Pub. No.: US 2015/0079075 A1
(43) Pub. Date: Mar. 19, 2015

(54) Anti-CD11A Antibodies and Uses Thereof

(71) Applicants: Richard Trauger, Leucadia, CA (US); Robert Fleischaker, Carlsbad, CA (US)

(72) Inventors: Richard Trauger, Leucadia, CA (US); Robert Fleischaker, Carlsbad, CA (US)

(73) Assignee: CytoDyn Inc., Vancouver, WA (US)

(21) Appl. No.: 14/344,697

(22) PCT Filed: Sep. 13, 2012
(86) PCT No.: PCT/US12/55132

§ 371 Date: Dec. 5, 2014

Related U.S. Application Data
(60) Provisional application No. 61/534,942, filed on Sep. 15, 2011.

(51) Int. Cl. C07K 16/28

(52) U.S. Cl. CPC C07K 16/2845; C07K 2317/92; C07K 2317/56
USPC 424/133.1; 530/387.3; 530/389.1; 530/391.1; 530/391.3; 424/172.1

(57) ABSTRACT

Provided herein are isolated human, chimeric, and humanized antibodies and antigen-binding fragments thereof that specifically bind to CD11a. Also provided are methods of treating human immunodeficiency virus (e.g., reducing the risk of developing or preventing the development of HIV infection or AIDS) in a subject that has an HIV infection or AIDS that include administering at least one of the antibodies or antigen-binding fragments to the subject. Also provided are methods of crosslinking CD11a on the surface of a cell that include contacting the cell with at least one of the antibodies or antigen-binding fragments. Also provided are compositions (e.g., pharmaceutical compositions) containing at least one of the antibodies or antigen-binding fragments.

### CROSS-REFERENCE TO RELATED APPLICATIONS

This application claims prior to U.S. Provisional Patent Application Ser. No. 61/534,942, filed Sep. 15, 2011, the entire contents of which is herein incorporated by reference.

### TECHNICAL FIELD

This invention relates to anti-CD11a antibodies and fragments thereof, compositions containing these antibodies and fragments, and methods of using these anti-antibodies and fragments.

### BACKGROUND

Lymphocyte function-associated antigen-1 (LFA-1) is a protein heterodimer that is involved in leukocyte adhesion during cellular interactions that play an essential role in immunological responses and inflammation in mammals (Larson et al., Immunol. Rev. 114:181-217, 1990). LFA-1 is a member of the β2 integrin family and is a heterodimer of the α subunit, CD11a, and the β subunit, CD18. CD18 is common to other β2 integrin family receptors (e.g., Mac01 and p150,95). The ligands of LFA-1 include intracellular adhesion molecule-1 (ICAM-1) which is expressed in the plasma membrane of leukocytes, endothelial cells, and dermal fibroblasts (Dustin et al., J. Immunol. 137:245-254, 1986), ICAM-2 which is expressed in the plasma membrane of endothelial cells and resting lymphocytes (de Fougerolles et al., J. Exp. Med. 174:253-267, 1991), and ICAM-3 which is expressed in the plasma membrane of monocytes and resting lymphocytes (de Fougerolles et al., J. Exp. Med. 179:619-629, 1994).

Monoclonal antibodies against LFA-1 have been shown, in vitro, to inhibit several T-cell-dependent immune functions including T-cell activation (Kuypers et al., Res. Immunol. 140:461, 1989), T-cell dependent B-cell proliferation (Fischer et al., J. Immunol. 136:3198-3203, 1986), target cell lysis (Krensky et al., J. Immunol. 131:611-binding site. The administration of an anti-LFA-1 antibody that binds to the ICAM-1 binding site on CD11a (in the LFA-1 heterodimer) can induce detrimental immunosuppression in a subject (e.g., a pathological reduction in T-cell activation and T-cell dependent B-cell proliferation).

The Human Immunodeficiency Virus (HIV) is an enveloped virus that acquires LFA-1 and ICAM-1 upon budding from the infected cell (Ott, Rev. Med Virol. 18(3):159-75, 2010). It has been proposed that the presence of these antigens in the viral envelope facilitates the transfer of viral RNA through the cellular membrane into the target cell through what has been termed the virological synapse (Gaia Vasiliver-Shami et al., Viruses 2:1239-1260, 2010). These cellular derived antigens on the viral particle thus provide a target for neutralization of virus infection via antibodies that target either LFA-1 or ICAM-1 (Gomez and Hildreth, J. Virol. 69:4628-4632, 1995). However, inhibition of LFA-1 and ICAM-1 by monoclonal antibodies that target the binding site of LFA-1 for ICAM-1 can result in clinically relevant immunosuppression (MacKenzie et al., 2010, 10 (1) Clinical Lymphoma, Myeloma & Leukemia E14-E16, 2010). Thus, antibodies and agents that target CD11a on cell and HIV and do not induce detrimental immunosuppression in a subject are desired.

### SUMMARY

Provided herein are human, chimeric, and humanized antibodies and antigen-binding fragments thereof that specifically bind to CD11a (e.g., bind to CD11a on the surface of a CD8⁺ T-cell). These antibodies and antigen-binding fragments bind to an epitope in CD11a that does not overlap with the ICAM-1 binding site (in the LFA-1 heterodimer) and therefore, do not decrease T-cell-dependent immune functions in a subject (e.g., T-cell activation, T-cell dependent B-cell activation, and target cell lysis). Some of the antibodies and antigen-binding fragments (e.g., multimeric antibodies and antigen-binding fragments) described herein can crosslink CD11a (e.g., present in LFA-1) present on the surface of a cell (e.g., in the plasma membrane of a CD8⁺ T-cell). Also provided are methods of using these antibodies and antigen-binding fragments to treat a human immunodeficiency virus (HIV) infection in a subject and crosslink CD11a (e.g., present in LFA-1) present on the surface of a cell (e.g., a CD8⁺ T-cell).

Provided herein are isolated human, chimeric, or humanized antibodies or antigen-binding fragments thereof. These antibodies and antigen-binding fragments thereof bind competitively with an antibody produced by the hybridoma deposited at the American Type Culture Collection (ATCC) and designated as HB 9579. In some embodiments, the antibody or antigen-binding fragment thereof has a K_{D} for binding to CD11a (e.g., human CD11a) equal to or less than 5 nM (e.g., equal to or less than 2 nM or equal to or less than 1 nM). In some embodiments, the antibody or antigen-binding fragment thereof is capable of binding CD11a on the surface of a human cell (e.g., a human CD8⁺ T-cell). In some embodiments, the antibody or antigen-binding fragment thereof is capable of crosslinking CD11a (e.g., as part of LFA-1) on the surface of the human cell (e.g., a human CD8⁺ T-cell).

In some embodiments, the antigen-binding fragment thereof is an antigen-binding fragment selected from the group of: a Fab fragment, a F(ab′)₂ fragment, and a scFv fragment. In some embodiments, the antibody or antigen-binding fragment thereof contains a light chain variable domain comprising a contiguous sequence of at least 5 (e.g., at least 5, 6, 7, 8, 9, 10, 15, 20, 25, or 30) amino acids within the sequence of SEQ ID NO: 1, SEQ ID NO: 19, or SEQ ID NO: 20, or a heavy chain variable domain containing a contiguous sequence of at least 5 (e.g., at least 5, 6, 7, 8, 9, 10, 15, 20, 25, or 30) amino acids within the sequence of SEQ ID NO: 2, SEQ ID NO: 21, or SEQ ID NO: 22. In some embodiments, the antibody or antigen-binding fragment thereof contains a light chain variable domain containing a contiguous sequence of at least 5 (e.g., at least 5, 6, 7, 8, 9, 10, 15, 20, 25, or 30) amino acids within the sequence of SEQ ID NO: 1, and a heavy chain variable domain containing a contiguous sequence of at least 5 (e.g., at least 5, 6, 7, 8, 9, 10, 15, 20, 25, or 30) amino acids within the sequence of SEQ ID NO: 2. In some embodiments, the antibody or antigen binding fragment thereof contains a light chain variable domain comprising a contiguous sequence of at least 5 (e.g., at least 5, 6, 7, 8, 9, 10, 15, 20, 25, or 30) amino acids within the sequence of SEQ ID NO: 20, and a heavy chain variable domain containing a contiguous sequence of at least 5 (e.g., at least 5, 6, 7, 8, 9, 10, 15, 20, 25, or 30) amino acids within the sequence of SEQ ID NO: 21 or SEQ ID NO: 22. In some embodiments, the antibody or antigen-binding fragment thereof contains a contiguous sequence of at least five 5 (e.g., at least 5, 6, 7, 8, 9, 10, 15, 20, 25, or 30) amino acids within the sequence of SEQ ID NO: 20, and a heavy chain variable domain containing a contiguous sequence of at least five 5 (e.g., at least 5, 6, 7, 8, 9, 10, 15, 20, 25, or 30) amino acids within the sequence of SEQ ID NO: 22.

In some embodiments, the antibody or antigen-binding fragment thereof contains a light chain variable domain containing the sequence of SEQ ID NO: 1, or a heavy chain variable domain containing the sequence of SEQ ID NO: 2. In some embodiments, the antibody or antigen-binding fragment thereof contains a light chain variable domain containing the sequence of SEQ ID NO: 1, and a heavy chain variable domain containing the sequence of SEQ ID NO: 2.

In some embodiments, the antibody or antigen-binding fragment thereof contains a light chain containing the sequence of SEQ ID NO: 3 or a heavy chain containing the sequence of SEQ ID NO: 4. In some embodiments, the antibody or antigen-binding fragment thereof contains a light chain containing the sequence of SEQ ID NO: 3 and a heavy chain containing the sequence of SEQ ID NO: 4.

In some embodiments, the antibody or antigen-binding fragment thereof contains a light chain variable domain containing the sequence of SEQ ID NO: 20, and a heavy chain variable domain containing the sequence of SEQ ID NO: 21 or SEQ ID NO: 22. In some embodiments, the antibody or antigen-binding fragment thereof contains a light chain variable domain containing the sequence of SEQ ID NO: 20, and a heavy chain variable domain containing the sequence of SEQ ID NO: 22.

In some embodiments, the antibody or antigen-binding fragment thereof contains a heavy chain variable domain sequence containing one, two, or three complementary determining regions (CDRs) selected from the group of: SFGMH (SEQ ID NO: 13), YISSGSSTLHYADTVKG (SEQ ID NO: 14), and GSRNLSHRLLS (SEQ ID NO: 15), or a light chain variable domain sequence containing one, two, or three CDRs selected from the group of KASQDVSTAVA (SEQ ID NO: 16), WASTRHT (SEQ ID NO: 17), and QQHYTTPWT (SEQ ID NO: 18). In some embodiments, the antibody or antigen-binding fragment thereof contains a heavy chain variable domain sequence containing the CDRs of SFGMH (SEQ ID NO: 13), YISSGSSTLHYADTVKG (SEQ ID NO: 14), and GSRNLSHRLLS (SEQ ID NO: 15), and a light chain variable domain sequence containing the CDRs of KASQDVSTAVA (SEQ ID NO: 16), WASTRHT (SEQ ID NO: 17), and QQHYTTPWT (SEQ ID NO: 18). In some embodiments, the antibody or antigen-binding fragment contains a light chain variable domain containing the sequence of SEQ ID NO: 19 or SEQ ID NO: 20, or a heavy chain variable domain containing the sequence of SEQ ID NO: 21 or SEQ ID NO: 22. In some embodiments, the antibody or antigen-binding fragment contains a light chain variable domain containing the sequence of SEQ ID NO: 19 or SEQ ID NO: 20, and a heavy chain variable domain containing the sequence of SEQ ID NO: 21 or SEQ ID NO: 22.

In some embodiments, the antibody or antigen-binding fragment does not induce detrimental immunosuppression in a mammal administered the antibody or antigen-binding fragment thereof. In some embodiments, the antibody or antigen-binding fragment is covalently linked to a stabilizing moiety (e.g., a protein, a carbohydrate, or a polymer, or a combination thereof). In some embodiments, the antibody or antigen-binding fragment is labeled. In some embodiments, the antibody is a single-chain antibody or a multimeric antibody. Also provided are pharmaceutical compositions containing at least one (e.g., one, two, three, or four) antibody or antigen-binding fragment described herein.

Also provided are methods that utilize at least one (e.g., one, two, three, or four) of any of the antibodies or antigen-binding fragments described herein. For example, provided herein are methods of treating human immunodeficiency virus (HIV) infection or AIDS in a subject. These methods include administering a pharmaceutically effective amount of the antibody or antigen-binding fragment thereof that specifically binds to CD11a (e.g., any of the antibodies or antigen-binding fragments described herein) to a subject having an HIV infection. Some embodiments of these methods further include detecting a decrease (e.g., a significant, observable, or detectable decrease) in the severity, frequency, and/or duration of at least one (e.g., at least one, two, three, four, or five) symptom of HIV infection in the subject. Some embodiments of these methods further include detecting a decrease (e.g., a significant, observable, or detectable decrease) in HIV viral titer in the subject. Some embodiments of these methods further include detecting an increase (e.g., a significant, observable, or detectable increase) in the ratio of CD4⁺ T-cells to CD8⁺ T-cells in the subject. Some embodiments of these methods further include detecting an increase (e.g., a significant, observable, or detectable increase) in the number of CD4⁺ cells or the total number of T-cells in the subject. In some embodiments, a subject identified as being at risk of previous exposure to HIV or a subject identified as being at risk of future exposure to FIV is administered a pharmaceutically effective amount of at least one antibody or antigen-binding fragment thereof that specifically binds to CD11a (e.g., any of the antibodies or antigen-binding fragments described herein). Some embodiments of these methods include identifying a subject having an increased risk of previous exposure to HIV or identifying a subject having an increased risk of future exposure to HIV, and administering a pharmaceutically effective amount of at least one antibody or antigen-binding fragment thereof that specifically binds to CD11a (e.g., any of the antibodies or antigen-binding fragments described herein) to a subject that is identified as having an increased risk of previous exposure to HIV or a subject that is identified as having an increased risk of future exposure to HIV.

In some embodiments of any of the methods described herein, the at least one antibody or antigen-binding fragment thereof is administered intravenously, intraarterially, ocularly, orally, subcutaneously, intraperitoneally, or intramuscularly. In some embodiments, the at least one antibody or antigen-binding fragment thereof is administered intravenously. In some embodiments, the at least one antibody and/or antigen-binding fragment thereof is administered to the subject at least once a week (e.g., once a week, twice a week, three times a week, and four times a week). In some embodiments, the at least one antibody or antigen-binding fragment thereof is administered to the subject at least once a day (e.g., once a day, twice a day, or three times a day). In some embodiments of all of the methods described herein, the administering does not result in detrimental immunosuppression in the subject.

Also provided are methods of using any of the isolated antibodies or antibody fragments described herein in the manufacture of a medicament for treating human immunodeficiency virus (HIV) infection or AIDS in a subject. Also provided herein are isolated antibodies or antibody fragments for use in treating human immunodeficiency virus (HIV) infection or AIDS in a subject.

Also provided are methods of crosslinking CD11a (e.g., as part of LFA-1) on the surface of a cell (e.g., a CD8⁺ T-cell). These methods include contacting a cell expressing CD11a in the plasma membrane with at least one (e.g., one, two, three, or four) antibody or antigen-binding fragment that specifically binds to CD11a (e.g., at least one of the antibodies or antigen-binding fragments thereof described herein), where the at least one antibody or antigen-binding fragment mediates crosslinking of CD11a (e.g., as part of LFA-1) on the surface of the cell. In some embodiments, the cell is a CD8⁺ T-cell (e.g., a human CD8⁺ T-cell).

Also provided herein are methods of using any of the isolated antibodies or antibody fragments described herein in the manufacture of a medicament for crosslinking CD11a (e.g., as part of LFA-1) on the surface of a cell (e.g., a CD8+ T-cell). Also provided herein are isolated antibodies or antibody fragments for use in crosslinking CD11a (e.g., as part of LFA-1) on the surface of a cell (e.g., a CD8+ T-cell).

In some embodiments of the methods described herein, the antibody or antigen-binding fragment thereof crosslinks the CD11a present in the plasma membrane of the CD8⁺ T-cell (e.g., a human CD8⁺ T-cell). In any of the embodiments described herein, the CD8⁺ T-cell (e.g., a human CD8⁺ T-cell) can be in vitro or in vivo (e.g., in a human).

By the term “isolated” is meant a molecule that is separated from at least one contaminant (e.g., protein, nucleic acid, lipid, or carbohydrate, or combination thereof). In non-limiting examples, an isolated antibody or antigen-binding fragment is at least 80%, 85%, 90%, 91%, 92%, 93%, 94%, 95%, 96%, 97%, 98%, or 99% pure by weight.

By the term “antibody” is meant any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) of an antibody that is capable of specifically binding to an epitope. Non-limiting examples of antibodies include: monoclonal antibodies (e.g., including full-length antibodies and antigen-binding fragments thereof), polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes antibody conjugates (e.g., an antibody conjugated to a stabilizing moiety, a detectable moiety, or a therapeutic agent) and derivatives (e.g., bi-specific antibodies and single-chain antibodies).

By the term “antigen-binding fragment” is meant any portion of a full-length antibody that contains at least one variable domain (e.g., a variable domain of a human heavy or light chain immunoglobulin) that is capable of specifically binding to an antigen. Non-limiting examples of antibody fragments include Fab, Fab′, F(ab′)₂, and Fv fragments, diabodies, linear antibodies, and multi-specific antibodies formed from antibody fragments.

By the term “human antibody” is meant an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

By the term “chimeric antibody” is meant an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

By the term “humanized antibody” is a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (CDR) region residues of the recipient antibody are replaced by hypervariable (CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance.

In some embodiments, the humanized antibody will contain substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

By the term “single-chain antibody” is meant a single polypeptide that contains at least two immunoglobulin variable domains (e.g., a variable domain of a mammalian heavy or light chain immunoglobulin) that is capable of specifically binding to an antigen. Non-limiting examples of single-chain antibodies are described herein.

By the term “multimeric antibody” is meant an antibody that contains four or more (e.g., six, eight, or ten) immunoglobulin variable domains. In some embodiments, the multimeric antibody is able to crosslink one target molecule (e.g., one CD11a molecule) to at least one second target molecule (e.g., a second CD11a molecule) on the surface of a mammalian cell (e.g., a human CD8⁺ T-cell). In some embodiments, the multimeric antibody crosslinks one CD11a molecule present in LFA-1 to at least one second CD11a molecule present in LFA-1 on the surface of a CD8⁺ T-cell (e.g., a human CD8⁺ T-cell).

An antibody or antigen-binding fragment thereof “specifically binds” to a particular antigen (e.g., CD11a) when it binds to that antigen, but recognizes and binds to a lesser extent (e.g., does not recognize and bind) to other molecules in a sample. In some embodiments, an antibody or an antigen-binding fragment thereof selectively binds to an epitope with an affinity (K_{D}) equal to or less than 1×10⁻⁷ M (e.g., equal to or less than 1×10⁻⁸ M, equal to or less than 5×10⁻⁹M, equal to or less than 2×10⁻⁹ M, or equal to or less than 1×10⁻⁹ M) in phosphate buffered saline. The ability of an agent or molecule to specifically bind to an antigen or a molecule (e.g., CD11a) may be determined using any of the methods known in the art or those methods described herein.

By the phrase “binds competitively” is meant the binding of two molecules to substantially the same site (e.g., epitope) in a target molecule. In some embodiments, the two molecules (e.g., antibodies and/or antigen-binding fragments) bind to the same site (e.g., epitope) in a target molecule. In some embodiments, the two molecules (e.g., antibodies and/or antigen-binding fragments) bind to overlapping sites (e.g., epitopes) in a target molecule. In instances where two molecules bind competitively, the binding of one of the two molecules to the target molecule decreases (e.g., a detectable or measurable decrease) or prevents the binding of the second of the two molecules to the target molecule. Methods for determining or detecting the competitive binding of two molecules to a target molecule are known in the art and described herein (e.g., competitive binding assays (e.g., competitive enzyme-linked immunosorbent assays or BioCore experiments)).

By the phrase “surface of a cell” is meant a molecule (e.g., a protein, such as a receptor) that is integrated or bound to the plasma membrane of a cell (e.g., a human cell). In some embodiments, the molecule present on the surface of a cell is a receptor protein that has at least one transmembrane domain (e.g., CD11a).

By the phrase “decrease in the severity, frequency, or duration of at least one symptom” is meant a detectable or observable decrease in the intensity or clinical scoring of at least one symptom of a disease (e.g., HIV infection or AIDS) in a subject, a detectable or observable decrease in the recurrence of at least one symptom of disease (e.g., HIV infection or AIDS) in a subject, or a detectable or observable decrease in the duration of at least one symptom of a disease (e.g., HIV infection or AIDS) in a subject. Symptoms of a disease in a subject may be detected, observed, or scored by a health care or veterinary professional, or any other individual. A decrease in the severity, frequency, or duration of at least one symptom of a disease in a human receiving a treatment (e.g., administered at least one of any of the antibodies, antigen-binding fragments, or derivatives or conjugates thereof described herein) may be compared to the severity, frequency, or duration of at least one symptom of disease in a control subject (e.g., a subject having the same disease not receiving treatment or the same subject prior to treatment).

By the phrase “symptoms of HIV infection or AIDS” is meant any physical manifestation of an HIV infection or AIDS in a human subject that is measurable or detectable. One or more symptoms of an HIV infection or AIDS can be detected during physical examination by a health care professional. As is known in the art, some symptoms of HIV infection or AIDS are detectable following the performance of a clinical assay (e.g., the number or level of CD4⁺ T-cells, the ratio of CD4⁺ T-cells to CD8⁺ T-cells, and the total number of T-cells or white blood cells in a subject). Non-limiting examples of symptoms of HIV infection or AIDS in a human include: fever, headache, muscle and joint pain, sore throat, rash, diarrhea, swollen lymph nodes (e.g., neck, axilla, or groin lymph nodes), night sweats, poor appetite, fatigue, weight loss, dry cough, shortness of breath, depression, peripheral neuropathy, confusion, changes in the level of consciousness, lesions on the tongue or in the mouth, and blurred or distorted vision.

The phrase “symptom of HIV infection or AIDS” also includes the presentation of one or more opportunistic infections (e.g., bacterial, viral, fungal, and/or parasitic infection) in a subject. An opportunistic infection typically is not observed in human subjects with healthy immune systems (immunocompetent subjects). Non-limiting examples of opportunistic infections include: cytomegalovirus (CMV) infection, Pneumocystis carinii pneumonia (PCP) (Pneumocystis jirovecii), Mycobacterium tuberculosis infection, recurrent pneumonia, Candidiasis infection (thrush), coccidiomycosis, cryptococcosis, herpes simplex virus infection (e.g., herpes simplex virus-1 infection), Salmonella infection, Shigella infection, Listeria infection, Campylobacter infection, cryptosporidiosis, microsporidiosis, Myocobacterium avium complex (MAC or MAI), astorvirus, histoplasmosis, isosporiasis, adenovirus infection, rotavirus infection, Clostridium difficile infection, toxoplasmosis (Toxoplasma gondii), Cryptococcus neoformans infection, and Penicillium marneffei infection.

By the term “complementary determining region” or “CDR” is meant a region within an immunoglobulin (heavy or light chain immunoglobulin) that forms part of an antigen-binding site in an antibody or antigen-binding fragment thereof. As is known in the art, a heavy chain immunoglobulin contains three CDRs: CDR1, CDR2, and CDR3, respectively, and a light chain immunoglobulin contains three CDRs: CDR1, CDR2, and CDR3. In any antibody or antigen-binding fragment thereof, the three CDRs from the heavy chain immunoglobulin and the three CDRs from the light chain immunoglobulin together form an antigen-binding site in an antibody or antigen-binding fragment thereof. The Kabat Database is one system used in the art to number CDR sequences present in a light chain immunoglobulin or a heavy chain immunoglobulin. One or more CDRs may also be present in antibody derivatives or conjugates (e.g., the antibody derivatives and conjugates described herein).

By the term “detrimental immunosuppression” is meant a nearly complete suppression of CD8⁺ T-cell activation (cytotoxic T-cell activation) in a subject following a therapeutic treatment.

By the term “viral titer” is meant the number of viral particles per unit of measurement (e.g., mL or L of plasma). Methods for determining the viral titer of HIV are known in the art.

By the term “stabilizing moiety” is meant a molecule that is covalently attached to an antibody or antigen-binding fragment thereof (e.g., any of the antibodies or antigen-binding fragments described herein) that increases (e.g., a significant or detectable increase) the half-life or preserves the antigen-binding activity of the antibody or antigen-binding fragment in vitro or in vivo. In non-limiting examples, a stabilizing moiety can be a protein, a nucleic acid, a polymer, or a carbohydrate (e.g., polysaccharide), or a combination thereof. In some embodiments, the stabilizing moiety increases the half-life or preserves the antigen-binding activity of the antibody or antigen-binding fragment in vitro (e.g., in a lyophilized powder or in solution). In some embodiments, the stabilizing moiety increases the half-life or preserves the antigen-binding activity of an antibody or antigen-binding fragment in vivo (e.g., in a human following administration).

By the term “HIV infection” is meant the presence of at least one HIV virion in a subject, a detectable level of HIV virions in a subject, or the presence or a detectable level of HIV genomic nucleic acid in a subject. Non-limiting examples of methods for detecting the presence or the levels of HIV virions or HIV genomic nucleic acid in a subject are known in the art and are described herein. For examples, methods for detecting or measuring the level of an HIV genomic nucleic acid in a subject are known in the art (e.g., reverse transcriptase polymerase chain reaction). Methods for detecting the presence of an HIV virion indirectly are also known in the art (e.g., measurement of anti-HIV antibody titers in a subject).

By the term “crosslinking CD11a” is meant the non-covalent binding or association of at least two different molecules of CD11a on the surface of a cell (e.g., in the plasma membrane of a CD8⁺ T-cell) mediated by a molecule that specifically binds to at least two different molecules of CD11a (e.g., at least one of the antibodies or antigen-binding fragments described herein). In some embodiments, the at least two molecules of CD11a that are crosslinked are present in LFA-1 (a heterodimer of CD11a and CD18).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows the amino acid sequence of the CD11a extracellular domain (SEQ ID NO: 11) and the amino acid sequence of a fragment of the CD11a extracellular domain containing the epitope recognized by the antibodies and antibody fragments described herein (SEQ ID NO: 12).

FIG. 2 shows a chimeric light chain immunoglobulin sequence (top sequence; SEQ ID NO: 5), an optimized mouse anti-CD11a light chain variable domain sequence (solid underline in the top sequence and the middle sequence; SEQ ID NO: 1), and the mouse anti-CD11a light chain variable domain sequence with a human immunoglobulin κ light chain constant domain (bottom sequence; SEQ ID NO: 3). The individual CDRs in the light chain variable domain are indicated in bold and solid underline (SEQ ID NOS: 16-18).

FIG. 3 shows a map of the GS50626-1 pFUSEss-CLIg-hk-muS6F1VL vector containing a sequence encoding the optimized mouse anti-CD11a light chain variable domain sequence and the human immunoglobulin κ light chain constant domain (both indicated in the vector as “muS6F1VL”).

FIG. 4 shows the complete nucleic acid sequence of the GS50626-1 pFUSEss-CLIg-hk-muS6F1VL vector (SEQ ID NO: 6) containing a sequence encoding a mouse anti-CD11a light chain variable domain sequence (underlined sequence) and a human immunoglobulin κ light chain constant domain.

FIG. 5 shows a chimeric heavy chain immunoglobulin sequence (top sequence; SEQ ID NO: 4) and an optimized mouse anti-CD11a heavy chain variable domain sequence (bottom sequence; SEQ ID NO: 2). The individual CDRs in the heavy chain variable domain are indicated in bold and solid underline (SEQ ID NOS: 13-15).

FIG. 6 shows a map of the GS50626-2 pFUSE-CHIg-hG1-muS6F1VH vector containing a sequence encoding an optimized mouse anti-CD11a heavy chain variable domain sequence and a human immunoglobulin IgG heavy chain constant domain (both indicated in the vector as “muS6F1VH”).

FIG. 7 shows the complete nucleic acid sequence of the GS50626-2 pFUSE-CHIg-hG1-muS6F1VH vector (SEQ ID NO: 7) containing a sequence encoding a mouse anti-CD11a heavy chain variable domain sequence (underlined sequence) and a human immunoglobulin IgG heavy chain constant domain.

FIG. 8 shows surface plasmon resonance (SPR) data for the binding of a chimeric antibody (chimeric light chain of SEQ ID NO: 3 and chimeric heavy chain of SEQ ID NO: 4) to the extracellular domain of CD11a (SEQ ID NO: 11).

FIG. 9 shows two additional optimized mouse anti-CD11a light chain variable domain sequences (huS6F1VL3 and huS6F1VL4; SEQ ID NO: 19 and 20), with the CDRs in bold and underlined (SEQ ID NOS: 16-18).

FIG. 10 shows two additional optimized mouse anti-CD11a heavy chain variable domain sequences (huS6F1VH1 and huS6F1VH4; SEQ ID NO: 21 and 22), with the CDRs in bold and underlined (SEQ ID NOS: 13-15).

### DETAILED DESCRIPTION

Provided herein are human, chimeric, or humanized antibodies and antigen-binding fragments that specifically bind to substantially the same epitope (the same or an overlapping epitope in CD11a) that is bound by the antibody produced from the hybridoma deposited at ATCC and designated HB 9579. The antibodies and antigen-binding fragments described herein do not detrimentally block or prevent LFA-1 binding to ICAM-1 and thus, do not cause detrimental immunosuppression when administered to a subject (e.g., a human). The antibodies and antigen-binding fragments described herein bind to an epitope that does not overlap the ICAM-1 binding site on CD11a. The specific epitope on CD11a, and antibodies and antigen-binding fragments that bind to this epitope are described herein. Also provided are pharmaceutical compositions that contain these antibodies and antigen-binding fragments, and methods of using these antibodies, antigen-binding fragments, and compositions.

### Epitope

Human CD11a is encoded by two different mRNA transcripts (NCBI Accession Nos. NM_{—}001114380.1 and NM_{—}002209.2), each transcript encodes a different isoform of CD11a (isoform a and isoform b). Isoform a (NCBI Accession No. NP_{—}002200.2) is a 1,170-amino acid protein containing a 25-amino acid signal sequence (amino acids 1-25 of NCBI Accession No. NP_{—}002200.2). The signal sequence is removed to yield the mature form of the protein (e.g., amino acids 26-1170 of NCBI Accession No. NP_{—}002200.2). Isoform b (NCBI Accession No. NP_{—}001107852.1) is a 1,086-amino acid protein containing a 25-amino acid signal sequence (amino acids 1-25 of NCBI Accession No. NP_{—}001107852.1). The signal sequence is removed to yield the mature form of the protein (e.g., amino acids 26-1086 of NCBI Accession No. NP_{—}001107852.1). A commercial source of a nucleic acid encoding human CD11a is available from Open Biosystems (clone MSH1011-61048). The mature forms of CD11a (isoform a and b) are transmembrane proteins located in the plasma membrane.

The antibodies and antigen-binding fragments described herein bind to a unique epitope in CD11a that does not overlap with the ICAM-1 binding site. The antibodies and antigen-binding fragments described herein bind to substantially the same epitope (e.g., the same epitope or an overlapping epitope) on CD11a that is recognized by the antibody produced from the hybridoma deposited at the ATCC and designated as HB 9579. The epitope recognized by the antibodies and antigen-binding fragments described herein was produced recombinantly as described in the methods below.

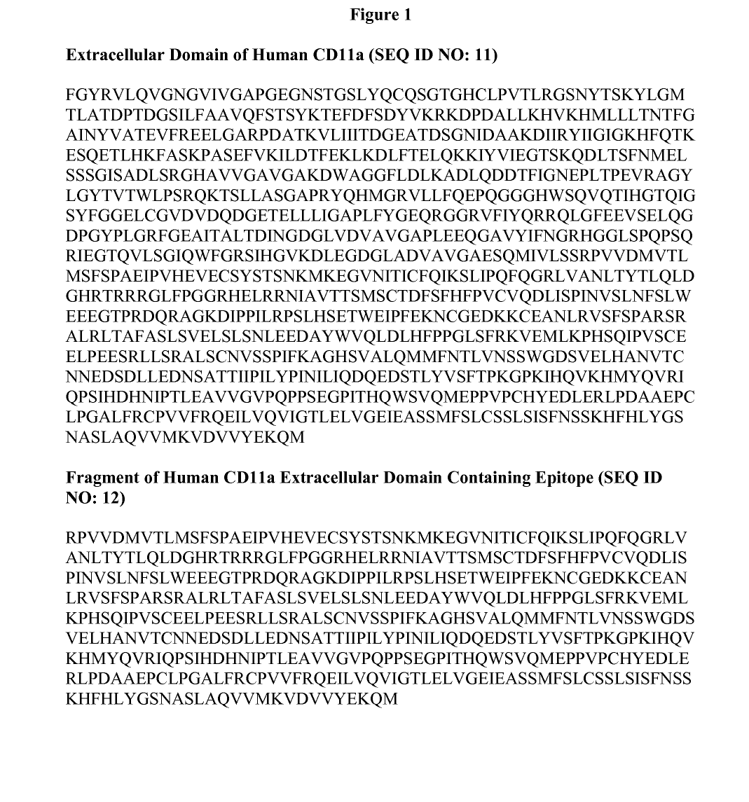

Two different portions of the nucleic acid sequence encoding CD11a were amplified from clone MHS1011-61048 from Open Biosystems to recombinantly produce the peptide CD11a epitope or a protein containing the CD11a epitope. To produce a protein containing the CD11a epitope, a nucleic acid sequence encoding the extracellular domain of CD11a (containing the peptide CD11a epitope recognized by the antibodies and antigen-binding fragments described herein) was amplified. This sequence was amplified using the forward extracellular domain primer ATATCATATGTACAAC CTGGACGTGCGGGGCGCGCGG (SEQ ID NO: 8) and the reverse primer ATATGCG GCCGCCATCTGCTTCTCATACACCACG TCAACC (SEQ ID NO: 9). The amino acid sequence of the extracellular domain of CD11a is shown in FIG. 1 (SEQ ID NO: 11).

To produce a shorter peptide containing the CD11a epitope recognized by the antibodies and antigen-binding fragments described herein, a nucleic acid sequence encoding the peptide CD11a epitope was amplified using the forward epitope primer ATATCATATGTCCCGGCCCGT GGTGGATATGGTCACC (SEQ ID NO: 10) and the reverse primer ATATGCGGCC GCCATCTGCTTCTCATACACCACG TCAACC (SEQ ID NO: 9). The amino acid sequence of the fragment of the human CD11a extracellular domain containing the epitope recognized by the antibodies and the antibody fragments described herein is shown in FIG. 1 (SEQ ID NO: 12).

The nucleic acid sequence encoding the extracellular domain of CD11a (SEQ ID NO: 11) and the nucleic acid sequence encoding the peptide CD11a epitope (SEQ ID NO: 12) were spliced into plasmids, which were used to transfect E. coli. The cloned plasmids were isolated from the E. coli and sequenced to confirm that the correct nucleic acid sequence had been amplified and cloned (i.e., the nucleic acid sequence encodes a polypeptide containing of SEQ ID NO: 11 or SEQ ID NO: 12).

The above plasmids were purified and the specific sequence encoding the extracellular domain of CD11a (SEQ ID NO: 11) or the sequence encoding the peptide CD11a antigen (SEQ ID NO: 12) were removed by digestion with the appropriate restriction enzymes, and the resulting nucleic acid sequences were inserted into PET-28 expression plasmids (EMD Biosciences). The PET-28 expression plasmid contains sequences that add a poly-His tag on the C-terminus of the protein encoded by the nucleic acid insert (e.g., SEQ ID NO: 11 or SEQ ID NO: 12). Each resulting PET-28 expression plasmid was used to transfect the E. coli strain BL-21, and clones were isolated and the presence of each plasmid was confirmed using gel electrophoresis.

Specific clones, identified as containing the specific PET-28 expression plasmid, were grown in shaker flasks. Isopropyl β-D-1-thiogalactopyranoside (IPTG) was added to the bacterial cell culture in order to induce protein expression from the PET-28 expression plasmid. The cells were harvested and aliquoted into 4 samples. One aliquot (from approximately 25 mL of culture) was lysed by enzymes (e.g., lysozyme) and detergents using methods known in the art. The clarified supernatant was loaded on a HiTrap Metal Chelating column (previously equilibrated with Ni), washed with a high salt solution, and eluted with imidazole. Approximately 2 mg of protein was isolated from the aliquot and antigenicity of the purified protein was confirmed by immuno-dot blot using methods known in the art. Western blots did not show reactivity of either of the proteins with the monoclonal antibody produced from the hybridoma deposited at the ATCC and designated HB 9579. However, the purified native peptide epitope or the purified native extracellular domain of CD11a (containing the peptide CD11a epitope specifically recognized by the antibodies and antigen-binding fragments described herein) were recognized by the monoclonal antibody produced from the hydridoma deposited at the ATCC and designated HB 9579. The extracellular domain of CD11a (SEQ ID NO: 11) or the shorter peptide containing the CD11a epitope (SEQ ID NO: 12) can be used to immunize mammals to produce additional antibodies or fragments (e.g., antibodies or antibody fragments described herein), or may be used to screen phage display libraries to identify additional antibodies and antigen-binding fragments (e.g., antibodies or antibody fragments described herein). These peptides can also be used to screen derivatives and conjugates of the antibodies or antigen-binding fragments described herein for the ability to bind to the same (or an overlapping) epitope recognized by the monoclonal antibody produced from the hybridoma deposited at the ATCC and designated HB 9579.

### Antibodies

Provided herein are antibodies and antigen-binding fragments thereof that specifically bind to an epitope on human CD11a that is substantially the same epitope (the same or an overlapping epitope in CD11a) that is bound by the antibody produced from the hybridoma deposited at ATCC and designated HB 9579. The antibodies and antigen-binding fragments described herein are capable of binding to CD11a on the surface of a cell, but do not inhibit the binding of CD11a (as part of LFA-1) to ICAM-1. For example, the antibodies and antigen-binding fragments described herein can bind to an epitope that does not overlap with the ICAM-1 binding site on CD11a (as part of LFA-1). As a result of this unique binding activity, the antibodies and antigen-binding fragments described herein do not induce detrimental immunosuppression in a subject following the administration of a therapeutically effective dose of these antibodies and/or antigen-binding fragments to a subject.

Some of the antibodies and antigen-binding fragments thereof described herein (e.g., multimeric antibodies or antigen-binding fragments thereof) can also induce the crosslinking of at least two different molecules of CD11a (e.g., present in LFA-1) on the surface of a cell. In some embodiments, this crosslinking of CD11a occurs on the surface of a CD8⁺ T-cell.

Methods for determining the ability of an antibody or antigen-binding fragment thereof to bind to an epitope on human CD11a (e.g., the unique epitope described herein) may be performed using the methods described herein and methods known in the art. Non-limiting examples of such methods include competitive binding assays using antibodies known to bind to the unique human CD11a epitope (e.g., the mouse monoclonal antibody produced by the hybridoma deposited at the ATCC and designated as HB 9579, and the exemplary chimeric antibodies described in the examples), such as enzyme-linked immunosorbent assays, BioCoRE®, affinity columns, immunoblotting, or protein array technology. In some embodiments, the binding activity of the antibody or antigen-binding fragment thereof is determined by contacting a human cell (e.g., a CD8⁺ T-cell, a dendritic cell, a fibroblast, or an epithelial cell) with the antibody or antigen-binding fragment thereof. In some embodiments, the binding activity of the antibody or antigen-binding fragment thereof is determined by contacting a mammalian (e.g., human) cell recombinantly overexpressing the CD11a epitope (e.g., full-length human CD11a or both full-length human CD11a and CD18). In some embodiments, the binding activity of the antibody or antigen-binding fragment thereof is determined by contacting the purified peptide epitope of CD11a (SEQ ID NO: 12) or a purified extracellular domain of CD11a (SEQ ID NO: 11) containing the epitope with the antibody or antigen-binding fragment thereof.

In some embodiments of any of the methods described herein, the antibody or antigen-binding fragment thereof binds to an epitope on human CD11a with an K_{D} equal to or less than 1×10⁻⁷M, a K_{D} equal to or less than 1×10⁻⁸ M, a K_{D} equal to or less than 5×10⁻⁸ M, a K_{D} equal to or less than 5×10⁻⁹M, a K_{D} equal to or less than 2×10⁻⁹ M, or a K_{D} equal to or less than 1×10⁻⁹ M under physiological conditions (e.g., in phosphate buffered saline).

An antibody is any antigen-binding molecule that contains at least one (e.g., one, two, three, four, five, or six) CDR (e.g., any of the three CDRs from an immunoglobulin light chain variable domain or any of the three CDRs from an immunoglobulin heavy chain variable domain) of an antibody that is capable of specifically binding to an epitope (e.g., the same or an overlapping epitope that is specifically recognized by an antibody produced by the hybridoma deposited at the ATCC and designated HB 9579), such as immunoglobulin molecules (e.g., light or heavy chain immunoglobulin molecules) and immunologically-active (antigen-binding) fragments of immunoglobulin molecules. For example, any of the antibodies or antigen-binding fragments of antibodies described herein can contain one, two, or three heavy chain variable domain CDRs selected from the group of: SFGMH (SEQ ID NO: 13), YISSGSSTLHYADTVKG (SEQ ID NO: 14), and GSRNLSHRLLS (SEQ ID NO: 15), and/or one, two, or three light chain variable domain CDRs selected from the group of: KASQDVSTAVA (SEQ ID NO: 16), WASTRHT (SEQ ID NO: 17), and QQHYTTPWT (SEQ ID NO: 18). In some embodiments, the antibodies or antigen-binding fragments thereof described herein can contain the three heavy chain variable domain CDRs of SFGMH (SEQ ID NO: 13), YISSGSSTLHYADTVKG (SEQ ID NO: 14), and GSRNLSHRLLS (SEQ ID NO: 15), and the three light chain variable domain CDRs of KASQDVSTAVA (SEQ ID NO: 16), WASTRHT (SEQ ID NO: 17), and QQHYTTPWT (SEQ ID NO: 18).

In some embodiments, an antibody or an antigen-binding fragment described herein can contain a heavy chain variable domain containing one, two, or three of: the CDR of SEQ ID NO: 16 with one or two amino acid insertions (within SEQ ID NO: 16 or at one or both terminal ends of SEQ ID NO: 16), deletions (within SEQ ID NO: 16 or at one or both terminal ends of SEQ ID NO: 16), or substitutions (within SEQ ID NO: 16 or at one or both terminal ends of SEQ ID NO: 16); the CDR of SEQ ID NO: 17 with one or two amino acid insertions (within SEQ ID NO: 17 or at one or both terminal ends of SEQ ID NO: 17), deletions (within SEQ ID NO: 17 or at one or both terminal ends of SEQ ID NO: 17), or substitutions (within SEQ ID NO: 17 or at one or both terminal ends of SEQ ID NO: 17); or the CDR of SEQ ID NO: 18 with one or two amino acid insertions (within SEQ ID NO: 18 or at one or both terminal ends of SEQ ID NO: 18), deletions (within SEQ ID NO: 18 or at one or both terminal ends of SEQ ID NO: 18), or substitutions (within SEQ ID NO: 18 or at one or both terminal ends of SEQ ID NO: 18); and/or light chain variable domain containing one, two, or three of: the CDR of SEQ ID NO: 13 containing one or two amino acid insertions (within SEQ ID NO: 13 or at one or both terminal ends of SEQ ID NO: 13), deletions (within SEQ ID NO: 13 or at one or both terminal ends of SEQ ID NO: 13), or substitutions (within SEQ ID NO: 13 or at one or both terminal ends of SEQ ID NO: 13); the CDR of SEQ ID NO: 14 containing one or two amino acid insertions (within SEQ ID NO: 14 or at one or both terminal ends of SEQ ID NO: 14), deletions (within SEQ ID NO: 14 or at one or both terminal ends of SEQ ID NO: 14), or substitutions (within SEQ ID NO: 14 or at one or both terminal ends of SEQ ID NO: 14); or the CDR of SEQ ID NO: 15 containing one or two amino acid insertions (within SEQ ID NO: 15 or at one or both terminal ends of SEQ ID NO: 15), deletions (within SEQ ID NO: 15 or at one or both terminal ends of SEQ ID NO: 15), or substitutions (within SEQ ID NO: 15 or at one or both terminal ends of SEQ ID NO: 15). Non-limiting examples of amino acid substitutions that can be made within a specific CDR sequence (e.g., any one or more of SEQ ID NOS: 13-18) or at one or both ends of a specific CDR sequence (e.g., any one or more of SEQ ID NOS: 13-18) are described herein (e.g., conservative or non-conservative amino acid substitutions).

An antibody can also be a single-chain antibody (e.g., as described herein). An antibody can be a whole antibody molecule (e.g., a human, humanized, or chimeric antibody) or a multimeric antibody (e.g., a bi-specific antibody).

Antibodies and antibody fragments as referred to herein include variants (including derivatives and conjugates) of antibodies or antibody fragments and multi-specific (e.g., bi-specific) antibodies or antibody fragments. Examples of antibodies and antigen-binding fragments thereof include, but are not limited to: single-chain Fvs (sdFvs), Fab fragments, Fab′ fragments, F(ab′)₂, disulfide-linked Fvs (sdFvs), Fvs, and fragments containing either a VL or a VH domain. The term “single chain Fv” or “scFv” as used herein refers to a polypeptide comprising at least one VL domain of an antibody linked to at least one VH domain of an antibody.

Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG₁, IgG₂, IgG₃, IgG₄, IgA₁, and IgA₂), or subclass. In some embodiments, the antibody or antigen-binding fragment thereof is an IgG₁ antibody or antigen-binding fragment thereof. In other embodiments, the antibody or antigen-binding fragment thereof is an IgG₄ antibody or antigen-binding fragment thereof. Immunoglobulins may have both a heavy and light chain.

An isolated fragment of human CD11a (e.g., the CD11a peptides described herein) or LFA-1 can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to a protein that is immunogenic in the species to be immunized, e.g., keyhole limpet hemocyanin, serum albumin, bovine thyroglobulin, or soybean trypsin inhibitor using a bifunctional or derivitizing agent, for example, malimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Animals can be injected with the antigenic peptide or protein more than one time (e.g., twice, three times, or four times).

Two exemplary fragments of human CD11a polypeptide that may be used to generate polyclonal or monoclonal antibodies are described herein (the extracellular domain of human CD11a (SEQ ID NO: 11) and a peptide containing the peptide epitope of CD11a (SEQ ID NO: 12) that is specifically recognized by the antibody produced by the hybridoma deposited at the ATCC and designated HB 9579). The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of human CD11a and encompasses an epitope of the protein such that an antibody raised against the peptide forms a specific immune complex with the protein. The antigenic peptide of CD11a is desirably a peptide that is specifically recognized by the antibody produced by the hybridoma deposited at the ATCC and designated HB 9579 or any of the chimeric antibodies described in the examples. As described above, the full length sequence of human CD11a is known in the art (NCBI Accession Nos.: NP_{—}002200.2 and NP_{—}001107852.1).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human CD11a, such as the two exemplary antigenic CD11a peptides described herein). The preparation can further include an adjuvant, such as Freund's complete or incomplete adjuvant, or a similar immunostimulatory agent.

Polyclonal antibodies can be prepared as described above by immunizing a suitable subject with a CD11a polypeptide, or an antigenic peptide thereof (e.g., a CD11a peptide that contains substantially the same epitope (e.g., the same epitope or an overlapping epitope) that is recognized by the antibody produced from the hybridoma deposited at the ATCC and designated HB 9579 or any of the chimeric antibodies described in the examples) as an immunogen. The antibody titer in the immunized subject can be monitored over time by standard techniques, such as with an enzyme-linked immunosorbent assay (ELISA) using the immobilized CD11a polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, N.Y.). Hybridoma cells producing a monoclonal antibody are detected by screening the hybridoma culture supernatants for antibodies that bind the polypeptide or epitope of interest, e.g., using a standard ELISA assay.

As an alternative to preparing monoclonal antibody-secreting hybridomas, a monoclonal antibody directed against a polypeptide can be identified and isolated by screening a recombinant combinatorial immunoglobulin library (e.g., an antibody phage display library) with the polypeptide or a peptide fragment containing the epitope of interest. Kits for generating and screening phage display libraries are commercially available (e.g., the Pharmacia Recombinant Phage Antibody System, Catalog No. 27-9400-01; and the Stratagene SurfZAP* Phage Display Kit, Catalog No. 240612). Additionally, examples of methods and reagents particularly amenable for use in generating and screening an antibody display library can be found in, for example, U.S. Pat. No. 5,223,409; WO 92/18619; WO 91/17271; WO 92/2079; WO 92/15679; WO 93/01288; WO 92/01047; WO 92/09690; WO 90/02809; Fuchs et al., Bio/Technology 9:1370-1372, 1991; Hay et al., Hum. Antibod. Hybridomas 3:81-85, 1992; Huse et al., Science 246:1275-1281, 1989; and Griffiths et al., EMBO J. 12:725-734, 1993.

In some embodiments of any of the methods described herein, the antibodies or antigen-binding fragments are human antibodies, humanized antibodies, or chimeric antibodies that contain a sequence from a human antibody (e.g., a human immunoglobulin constant domain or human immunoglobulin variable domain framework regions). Humanized antibodies are chimeric antibodies that contain a minimal sequence derived from non-human (e.g., mouse) immunoglobulin. In some embodiments, a humanized antibody is a human antibody that has been engineered to contain at least one complementary determining region (CDR) present in a non-human antibody (e.g., a mouse, rat, rabbit, or goat antibody). In some embodiments, a humanized antibody or fragment thereof can contain all three CDRs of a light chain of a non-human antibody (e.g., the antibody produced from the hybridoma deposited at the ATCC and designated HB 9579 or any of the chimeric antibodies described in the examples) or a human antibody that specifically binds to substantially the same epitope (e.g., the same or an overlapping epitope) that is recognized by the antibody produced from the hybridoma deposited at the ATCC and designated HB 9579 or any of the chimeric antibodies described in the examples. In some embodiments, the humanized antibody or fragment thereof can contain all three CDRs of a heavy chain of a non-human monoclonal antibody (e.g., the mouse monoclonal antibody produced by the hybridoma deposited with the ATCC and designated HB 9579 or any of the chimeric antibodies described in the examples) or a human antibody that specifically binds to substantially the same epitope (e.g., the same or an overlapping epitope) as the antibody produced by the hybridoma deposited with the ATCC and designated HB 9579 or any of the chimeric antibodies described in the examples. In some embodiments, the framework region residues of the human immunoglobulin are replaced by corresponding non-human (e.g., mouse) antibody residues. In some embodiments, the humanized antibodies can contain residues which are not found in the human antibody or in the non-human (e.g., mouse) antibody. Methods for making a humanized antibody from a non-human (e.g., mouse) monoclonal antibody are known in the art. Additional non-limiting examples of making a chimeric (e.g., humanized) antibody are described herein.

In some embodiments, the antibodies are chimeric antibodies that contain a light chain immunoglobulin that contains the light chain variable domain of a non-human antibody (e.g., a mouse antibody) or at least one CDR of a light chain variable domain of a non-human antibody (e.g., a mouse antibody) and the constant domain of a human immunoglobulin light chain (e.g., human κ chain constant domain). In some embodiments, the antibodies are chimeric antibodies that contain a heavy chain immunoglobulin that contains the heavy chain variable domain of a non-human (e.g., a mouse antibody) or at least one CDR of a heavy chain variable domain of a non-human (e.g., a mouse antibody) and the constant domain of a human immunoglobulin heavy chain (e.g., a human IgG heavy chain constant domain). In some embodiments, the chimeric antibodies contain a portion of a constant (Fc domain) of a human immunoglobulin.

In some embodiments, the antibodies or antigen-binding fragments thereof can be multi-specific (e.g., multimeric). For example, the antibodies can take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers within an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers. Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) (available, for example, from Pierce Biotechnology, Inc., Rockford, Ill.) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers (see, for example, WO 96/27011).

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents are well known in the art and are disclosed in U.S. Pat. No. 4,676,980, along with a variety of cross-linking techniques.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Additional methods have been developed to facilitate the direct recovery of Fab′-SH fragments from E. coli, which can be chemically coupled to form bi-specific antibodies. Shalaby et al. (J. Exp. Med. 175:217-225, 1992) describes the production of a fully-humanized bi-specific antibody F(ab′)₂ molecule. Each Fab′ fragment was separately secreted from E. coli and subjected to direct chemical coupling in vitro to form the bi-specific antibody.

Additional techniques for making and isolating bi-specific antibody fragments directly from recombinant cell culture have also been described. For example, bi-specific antibodies have been produced using leucine zippers (Kostelny et al., J. Immunol. 148:1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers.

The diabody technology described by Hollinger et al. (Proc. Natl. Acad. Sci. U.S.A. 90:6444-6448, 1993) is an additional method for making bi-specific antibody fragments. The fragments contain a heavy chain variable domain (V_{H}) connected to a light chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another method for making bi-specific antibody fragments by the use of single-chain Fv (sFv) dimers has been described in Gruber et al. (J. Immunol. 153:5368, 1994). Alternatively, the bi-specific antibody can be a “linear” or “single-chain antibody” produced using the methods described, for example, in Zapata et al. (Protein Eng. 8:1057-1062, 1995). In some embodiments the antibodies have more than two antigen-binding sites. For example, tri-specific antibodies can be prepared as described in Tutt et al. (J. Immunol. 147:60, 1991).

Alternatively, antibodies can be made to multimerize through recombinant DNA techniques. IgM and IgA naturally form antibody multimers through the interaction with the mature J chain polypeptide. Non-IgA or non-IgM molecules, such as IgG molecules, can be engineered to contain the J chain interaction domain of IgA or IgM, thereby conferring the ability to form higher order multimers on the non-IgA or non-IgM molecules (see, for example, Chintalacharuvu et al., Clin. Immunol. 101:21-31, 2001, and Frigerio et al., Plant Physiol. 123:1483-1494, 2000). IgA dimers are naturally secreted into the lumen of mucosa-lined organs. This secretion is mediated through the interaction of the J chain with the polymeric IgA receptor (pIgR) on epithelial cells. If secretion of an IgA form of an antibody (or of an antibody engineered to contain a J chain interaction domain) is not desired, it can be greatly reduced by expressing the antibody molecule in association with a mutant J chain that does not interact well with pIgR (Johansen et al., J. Immunol., 167:5185-192, 2001). ScFv dimers can also be formed through recombinant techniques known in the art. An example of the construction of scFv dimers is given in Goel et al. (Cancer Res. 60:6964-71, 2000). Antibody multimers may be purified using any suitable method known in the art, including, but not limited to, size exclusion chromatography.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

Any of the antibodies or antigen-binding fragments described herein may be conjugated to a label (e.g., a radioisotope, fluorophore, chromophore, or the like) or a therapeutic agent (e.g., a proteinaceous or small molecule therapeutic agent).

Non-limiting examples of antibodies or antigen-binding fragments thereof that can be used in any of the methods described herein are antibodies or antigen-binding fragments that contain at least five (e.g., at least 5, 10, 15, 20, 25, or 30) contiguous amino acids within the light chain variable domain sequence and/or at least five (e.g., at least 5, 10, 15, 20, 25, or 30) contiguous amino acids within the heavy chain variable domain sequence of the antibody produced from the hybridoma deposited with the ATCC and designated HB 9579 or any one of the chimeric antibodies described in the examples (e.g., heavy chain variable domain sequences of SEQ ID NO: 2, SEQ ID NO: 21, or SEQ ID NO: 22, or light chain variable domain sequences of SEQ ID NO: 1, SEQ ID NO: 19, or SEQ ID NO: 20). In some embodiments, the antibodies or antigen-binding fragments thereof that can be used in any of the methods described herein contain the light chain variable domain sequence and/or the heavy chain variable domain sequence of the antibody produced from the hybridoma deposited with the ATCC and designated HB 9579 or any one of the chimeric antibodies described in the examples (e.g., heavy chain variable domain sequences of SEQ ID NO: 2, SEQ ID NO: 21, or SEQ ID NO: 22, or light chain variable domain sequences of SEQ ID NO: 1, SEQ ID NO: 19, or SEQ ID NO: 20). In some embodiments, the antibodies or antigen-binding fragments thereof that can be used in any of the methods described herein contain light chain variable domain sequence and/or the heavy chain variable domain sequence of the antibody produced from the hybridoma deposited with the ATCC and designated HB 9579 or any of the chimeric antibodies described in the examples (e.g., heavy chain variable domain sequences of SEQ ID NO: 2, SEQ ID NO: 21, or SEQ ID NO: 22, or light chain variable domain sequences of SEQ ID NO: 1, SEQ ID NO: 19, or SEQ ID NO: 20), and further contain at least one F_{c} domain of a human antibody (e.g., a human light or heavy chain antibody). In some embodiments, the antibodies or antigen-binding fragments used in any of the methods described herein contain the light chain sequence of a chimeric antibody described in the examples (e.g., SEQ ID NO: 3) and/or contain the heavy chain sequence of the chimeric antibody described in the examples (e.g., SEQ ID NO: 4). In some embodiments, the antibodies or antigen-binding fragments used in any of the methods described herein contain the light chain variable domain sequence of SEQ ID NO: 1, SEQ ID NO: 19, or SEQ ID NO: 20, and/or contain the heavy chain variable domain sequence of SEQ ID NO: 2, SEQ ID NO: 21, or SEQ ID NO: 22. In some embodiments, the antibody or antigen-binding fragment thereof contains a light chain variable domain sequence of SEQ ID NO: 20 and a heavy chain variable domain containing the sequence of SEQ ID NO: 21 or SEQ ID NO: 22. In some embodiments, the antibody or antigen-binding fragment thereof contains a variable domain containing the sequence of SEQ ID NO: 20 and a heavy chain variable domain containing the sequence SEQ ID NO: 22. In some embodiments, the antibody is selected from one of the antibodies described in the examples. In some embodiments, the antigen-binding fragment is derived from one of the antibodies described in the examples.

Additional non-limiting examples of antibodies or antigen-binding fragments thereof that can be used in any of the methods described herein are antibodies or antigen-binding fragments that contain one, two, or three of the CDRs present in the light chain variable domain sequence and/or one, two, or three of the CDRs present in the heavy chain variable domain sequence of the antibody produced from the hybridoma deposited with the ATCC and designated HB 9579 or any of the chimeric antibodies described in the examples (light chain variable domain CDRs selected from SEQ ID NOS: 16-18, and heavy chain variable domain CDRs selected from SEQ ID NOS: 13-15).

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target epitope (e.g., the CD11a epitope recognized by the antibody produced by the hybridoma deposited with the ATCC and designated HB 9579 or any of the chimeric antibodies described in the examples). Any combination of deletions, insertions, and/or combinations can be made to arrive at an antibody or antigen-binding fragment thereof that has increased binding affinity for the target epitope. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

One useful method for identification of single amino acids or regions that are important for the specificity or strength (affinity) for binding to an epitope is called alanine scanning mutagenesis (Cunningham et al., Science 244:1081-1085, 1989). In this method a residue or a group of residues, such as arginine, aspartate, histidine, lysine, and glutamate, are replaced by a neutral or negatively-charged amino acid (e.g., alanine or polyalanine) to change the interaction of the amino acid with the CD11a antigen. The amino acid locations showing functional sensitivity to the substitutions are then refined by introducing further or other variants at, or near, the sites of substitution. While the site for introducing an amino acid sequence variation is predetermined, the nature of the mutation need not be predetermined. For example, alanine scanning mutagenesis or random mutagenesis can be performed at the target codon or region and the expressed antibodies or antigen-binding fragment variants can be screened for the desired activity (e.g., affinity or specificity for the target epitope).

Amino acid sequence insertions include amino- and/or carboxy-terminal fusions ranging in length from one residue to polypeptides containing a hundred or more residues, as well as intrasequence insertions of single or multiple amino acid residues. Examples of terminal insertions include an anti-CD11a antibody with an N-terminal methionyl residue or an antibody fused to an epitope tag. Other insertional variants of an anti-CD11a antibody include the fusion of an enzyme or a polypeptide which increases the serum half-life of the antibody (including the non-limiting examples described herein) to the N- or C-terminus of the anti-CD11a antibody or fragment.

Another type of variant is an amino acid substitution variant. These variants have at least one amino acid residue in the anti-CD11a antibody or fragment removed and a different residue inserted in its place. The sites of greatest interest for substitutional mutagenesis include the hypervariable loops (i.e., CDRs), but framework alterations can also be introduced. Non-limiting exemplary substitutions are: Ala substituted with Val, Leu, or Ile; Arg substituted with Lys, Gln, and Asn; Asn substituted with Gln, His, Lys, or Arg; Asp substituted with Glu; Cys substituted with Ser; Gln substituted with Asn; Glu substituted with Asp; Gly substituted with Pro or Ala; His substituted with Asn, Gln, Lys, or Arg; Ile substituted with Leu, Val, Met, Ala, Phe, or norleucine; Leu substituted with norleucine, Ile, Val, Met, Ala, or Phe; Lys substituted with Arg, Gln, or Asn; Met substituted with Leu, Phe, or Ile; Phe substituted with Leu, Val, Ile, Ala, or Tyr; Pro substituted with Ala; Ser substituted with Thr; Thr substituted with Ser; Trp substituted with Tyr or Phe; Tyr substituted with Trp, Phe, Thr, or Ser; and Val substituted with Ile, Leu, Met, Phe, Ala, or norleucine.

Hypervariable region residues or framework residues involved in antigen binding are generally substituted in a relatively conservative manner. If such substitutions results in a change in the biological activity (e.g., affinity of the antibody or fragment thereof to an epitope), then more substantial changes are introduced and the products screened for biological activity (e.g., affinity of the antibody or fragment thereof to an epitope). Substantial alterations in the biological properties of an antibody or antigen-binding fragment thereof are accomplished by selecting substitutions that differ significantly in their effect on maintaining (a) the structure of the polypeptide backbone in the area of the substitution, for example, a β-sheet or α-helix secondary structure, (b) the charge or hydrophobicity of the molecule at the target site, or (c) the bulk of the side chain. Naturally-occurring residues are divided into groups based on common side chain properties: hydrophobic residues (norleucine, Met, Ala, Val, Leu, and Ile); neutral hydrophilic residues (Cys, Ser, and Thr); acidic residues (Asp and Glu); basic residues (Asn, Gln, His, Lys, and Arg); residues that influence chain orientation (Gly and Pro); and aromatic residues (Tyr, Tyr, and Phe). Non-conservative substitutions will entail exchanging a member of one of these classes for another class.

Any cysteine residue not involved in maintaining the proper conformation of the anti-CD11a antibody or fragment can also be substituted, generally with serine, to improve the oxidative stability of the molecule and prevent aberrant crosslinking. Conversely, cysteine bond(s) can be added to the antibody or fragment to improve its stability (particularly where the antibody is an antibody fragment, such as an Fv fragment).

Another type of amino acid variant of an anti-CD11a antibody or fragment is one that alters the original glycosylation pattern of the antibody. By “altering the glycosylation pattern” is meant deleting one or more carbohydrate moieties found in the antibody or fragment, adding one or more glycosylation sites that are not present in the antibody or fragment, and/or changing the type of carbohydrate moiety attached at particular site in the antibody or fragment. Glycosylation of antibodies and antibody fragments is typically either N-linked or O-linked. N-linked refers to the attachment of the carbohydrate moiety to the side chain of an asparagine residue. The tripeptide sequences Asn-X-Ser and Asn-X-Thr, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars of N-aceylgalactosamine, galactose, or xylose to a hydroxyamino acid, most commonly serine or threonine, although 5-hydroxyproline may also be used.

Addition of glycosylation sites to the antibody can be accomplished by altering the amino acid sequence such that is contains one or more of the above-described tripeptide sequences (for N-linked glycosylation sites). The alteration may also be made by the addition of, or substitution by, one or more serine or threonine residues to the sequence of the original antibody (for O-linked glycosylation sites). Likewise, an N-linked glycosylation site may be changed to an O-linked glycosylation site, or a glycosylation site may be removed by mutagenesis.

Nucleic acid molecules encoding amino acid sequence variants of any of the anti-CD11a antibodies or antigen-binding fragments described herein can be prepared by a variety of methods known in the art. These methods include, but are not limited to, isolation from a natural source (in the case of naturally-occurring amino acid sequence variants) or preparation of oligonucleotide-mediated (or site-directed mutagenesis) PCR mutagenesis, and cassette mutagenesis of an earlier prepared variant or a non-variant version of an anti-CD11a antibody or antibody fragment.

Ordinarily, amino acid sequence variants of the human, humanized, or chimeric anti-CD11a antibody will contain an amino acid sequence having at least 75%, 80%, 85%, 90%, 95%, 96%, 97%, 98%, or 99% percent identity with a sequence present in the light or heavy chain of the original human, humanized, or chimeric antibody (e.g., any of SEQ ID NOS: 1-4 and 13-24). Identity or homology with respect to an original sequence is defined herein as the percentage of amino acid residues present within the candidate sequence that are identical with a sequence present within the human, humanized, or chimeric anti-CD11a antibody or fragment, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Additional modifications to the anti-CD11a antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-CD11a antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatizing agent that is capable of reacting with selected side chains or the N- or C-terminal residues.

Cysteine residues are most commonly are reacted with α-haloacetates (and corresponding amines), such as chloroacetic acid or choroacetamide, to give carboxymethyl or carboxyaminomethyl derivatives. Cysteine residues also are derivatized by reaction with bromotrifluoroacetone, α-bromo-β-(5-imidozyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidine residues can be derivatized by reaction with diethylpyrocarbonate at pH 5.5-7.0. Parabromophenacyl bromide is also useful; the reaction is typically performed in 0.1 M sodium cacodylate at pH 6.0.

Lysine and amino-terminal residues can be reacted with succinic or other carboxylic acid anhydrides. Derivatization with these agents has the effect of reversing the charge of the lysinyl residues. Other suitable reagents for derivatizing α-amino-containing residues include imidoesters, such as methyl picolinimidate, pyridoxal phosphate, pyridoxal, chloroborohydride, trinitrobenzenesulfonic acid, O-methylisourea, 2,4-pentadione, and transaminase-catalyzed reaction with glyoxylate.

Arginine residues can be modified by reaction with one or more conventional agents, such as phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents can react with the groups of lysine, as well as the arginine epsilon-amino group.

The modification of tyrosine residues can be made by reaction with aromatic diazonium compounds or tetranitromethane. Most commonly, N-acetylimidizole and tetranitromethane are used to form O-acetyl tyrosyl species and 3-nitro derivatives, respectively. For example, tyrosine residues in antibodies or antibody fragments can be iodinated using ¹²⁵I or ¹³¹I for use in radioimmunoassays.

Carboxy side groups (aspartic acid or glutamic acid) can be selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. In addition, aspartic acid and glutamic acid residues are converted to asparaginyl or glutaminyl residues by reaction with ammonium ions. Glutaminyl and asparaginyl residues are frequently deamidated to the corresponding glutamyl and aspartyl residues, respectively. These residues are deamidated under neutral or basic conditions. In some embodiments, antibodies and antigen-binding fragments can contain these deaminated forms of aspartic acid or glutamic acid.

Additional modifications that can be made include hydroxylation of proline or lysine, phosphorylation of the hydroxyl groups of serine or threonine residues, methylation of the α-amino groups of lysine, arginine, and histidine (T.E. Creighton, Proteins: Structure and Molecular Properties, W.H. Freeman & Co., San Francisco, pp. 79-86, 1983), acetylation of the N-terminal amine, and amidation of any C-terminal carboxyl group.

An additional type of covalent modification that can be made is the chemical or enzymatic coupling of a glycoside to an antibody or an antigen-binding fragment of an antibody. These procedures are advantageous in that they do not require production of the antibody or fragment in a host cell that has glycosylation capabilities for N- or O-linked glycosylation. Depending on the coupling mode used, the sugar(s) can be attached to (a) an arginine or histidine, (b) a free carboxyl group, (c) a free sulfhydryl group, (d) a free hydroxyl group, such as those of serine, threonine, or hydroxyproline, (e) an aromatic residue, such as phenylalanine, tyrosine, or tryptophan, or (f) the amide group of glutamine. Methods for coupling a glycoside to an antibody or antigen-binding fragment thereof are described in WO 87/05330 and Aplin et al. (CRC Crit. Rev. Biochem. 259-306, 1981).

Removal of any carbohydrate moieties present in an antibody can be accomplished chemically or enzymatically. Chemical deglycosylation requires exposure of the antibody to the compound trifluoromethanesulfonic acid or an equivalent compound. This treatment results in the cleavage of most or all sugars except the linking sugar N-acetylglucosamine or N-acetylgalactosamine, while leaving the antibody intact. Chemical deglycosylation is described in Hakimuddin et al. (Arch. Biochem. Biophys. 259:52, 1987) and Edge et al. (Anal. Biochem. 118:131, 1981). Enzymatic cleavage of carbohydrate moieties on antibodies can be achieved by the use of a variety of endo- and exo-glycosidases as described in Thotakura et al. (Methods Enzymol. 138:350, 1987).

In some embodiments, the covalent modification of an antibody or antigen-binding fragment thereof is the attachment of a variety of non-proteinaceous polymers, such as polyethylene glycol, polypropylene glycol, or polyoxylalkylenes. Methods for the attachment of nonproteinaceous polymers are known in the art and are described in U.S. Pat. Nos. 4,640,835; 4,496,689; 4,301,144; 4,670,417; 4,791,192; and 4,179,337.

### Pharmaceutical Compositions

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfate, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats HIV infection or AIDS in a subject (e.g., decreases the risk of developing or prevents the development of disease (e.g., an HIV infection or AIDS) in a subject (e.g., a human subject identified as being at risk of a future exposure to HIV (e.g., through participation in unprotected sexual intercourse, exposure to a blood product(s), or exposure to a needle(s) or a sharp object(s) contaminated with HIV), or a subject identified as being at risk of a previous exposure to HIV (e.g., a subject who has previously or currently participates in unprotected sexual intercourse, a subject who has been exposed to a blood product(s), or a subject who has been exposed to or punctured with a needle(s) or a sharp object(s) contaminated with HIV), decreases the severity, frequency, and/or duration of one or more symptoms of a disease (e.g., an HIV infection or AIDS) in a subject (e.g., a human), increases the ratio of CD4⁺ T-cells to CD8⁺ T-cells in a subject (e.g., a human) with an HIV infection or AIDS, increases the number of CD4⁺ T cells in a subject (e.g., a human) with an HIV infection or AIDS, and/or increases the total T-cell or white blood cell count in a subject (e.g., a human) with a disease (e.g., an HIV infection or AIDS) (e.g., as compared to a control subject having the same disease but not receiving treatment or the same subject prior to treatment)). The effectiveness and dosing of any of the antibodies or antigen-binding fragments described herein can be determined by a health care professional or veterinary professional using methods known in the art, as well as by the observation of one or more symptoms of disease (e.g., an HIV infection or AIDS) in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 ng/kg to about 500 mg/kg; about 100 ng/kg to about 500 mg/kg; about 100 ng/kg to about 50 mg/kg; about 10 ng/kg to about 5 mg/kg; about 10 ng/kg to about 0.5 mg/kg; or about 1 ng/kg to about 50 ng/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration.

### HIV and AIDS

Human immunodeficiency virus (HIV) is associated with a progressive catastrophic immunosuppressive disease in certain primates, including humans. Humans infected with HIV experience proliferation of a certain class of white blood cells known as cytotoxic T-lymphocytes (CTLs, also known as CD8⁺ T-cells). The final stage of HIV infection is commonly known as acquired immune deficiency syndrome (AIDS), and can result in a significant decrease in both CD4⁺ T-cells and CD8⁺ T-cells in some instances.

As is well known in the art, the clinical signs and symptoms of HIV infection and AIDS are primarily due to a profound loss of lymphocytes marked with the CD3 and CD4 antigens (CD4⁺ T-cells). It is also generally accepted that the infectious agent in AIDS is HIV. Non-limiting examples of symptoms of HIV infection within the first couple of weeks following contact with the virus include: fever, headache, sore throat, swollen lymph glands, and rash. Additional symptoms of HIV infection that can appear later include: mild infections, swollen lymph nodes, diarrhea, weight loss, fever, cough, and shortness of breath. HIV infection typically progresses to AIDS in about 10 years. By the time AIDS develops, the subject's immune system has been severely damaged. Later symptoms of HIV infection or AIDS include: soaking night sweats, shaking chills or fever higher than 38° C. for several weeks, cough or shortness of breath, chronic diarrhea, persistent white spots or unusual lesions on the tongue or in mouth, headaches, persistent unexplained fatigue, blurred or distorted vision, weight loss, and skin rashes or bumps.

Subjects having HIV infection or AIDS also have an increased risk of developing a number of opportunistic infections (e.g., bacterial, viral, fungal, and protozoal infections) and cancers. Non-limiting examples of opportunistic bacterial infections include bacterial strains that cause diarrhea (e.g., Salmonella sp., Campylobacter sp., Shigella sp.), bacterial strains that cause pneumonia (e.g., Streptococcus pneumonia, Haemophilus influenzae, Pseudomonas aeruginosa, Staphylococcus aureus, Legionella pneumophila, Mycoplasma pneumonia, and Chlamydia pneumoniae), Mycobacterium avium, Treponema pallidum, and Mycobacterium tuberculosis. Non-limiting examples of opportunistic viral infections include: cytomegalovirus (CMV), hepatitis C, herpes simplex virus (oral and genital herpes), herpes zoster virus (shingles), human papilloma virus (HPV), molluscum contagiosum virus (MCV), Epstein-Barr virus (EBV), and JC polyomavirus. Non-limiting examples of opportunistic fungal infections include: Aspergillus, Candida albicans, Coccidioides immitis, Coccidioides posadasii, Crytococcus neoformans, and Histoplasma capsulatum. Non-limiting examples of protozoal opportunistic infections include: Crytposporidium sp., Isospora belli, Microsporidium sp. (e.g., Enterocytozoon bieneusi), Pneumocystis jiroveci, and Toxoplasma gondii. Subjects with an HIV infection or AIDS also have an increased risk of developing a cancer, including but limited to: anal dysplasia or cancer, cervical dysplasia or cancer, Kaposi's sarcoma, and lymphomas. In addition, subjects having an HIV infection or AIDS also have a reduction in CD4⁺ T-cell levels, a decrease in the ratio of CD4+ T-cells to CD8+ T-cells, and/or a decrease in the total number of T-cells or white blood cells.

Methods for diagnosing a subject as having an HIV infection or AIDS are known in the art. For example, HIV infection can be diagnosed by a skilled medical professional (e.g., a physician, a nurse, a physician's assistant, or a laboratory worker) by the observation or detection of: one or more symptoms of an HIV infection or AIDS in a subject (e.g., any one or any combination of the symptoms described herein); detecting one or more opportunistic bacterial, fungal, viral, or parasitic infections (e.g., any one or more of the opportunistic bacterial, fungal, viral, or parasitic infections described herein); detecting a decrease in the level of CD4⁺ T-cells; detecting a decrease in the total levels of T-cells or white blood cells, detecting a decrease in the ratio of CD4⁺ T-cells to CD8⁺ T-cells; and/or detecting a cancer in a subject (e.g., any of the cancers described herein). Methods for diagnosing a subject as having an HIV infection or AIDS can also be performed using a diagnostic kit that detects the presence of an HIV protein or nucleic acid (e.g., HIV genomic nucleic acid) in a biological sample from the subject or detects the presence of antibodies that recognize an epitope in an HIV protein. Exemplary non-limiting HIV diagnostic kits include: Aware™ HIV-1/2 BSP (Calypte Biomedical), Bioline HIV 1/2 3.0 (Standard Diagnostics), Bionor™ HIV-1&2 (Bionor A/S), Bundi™ Rapid HIV 1/2 (Bundi International Diagnostics Ltd.), Calypte® Aware™ HIV-1/2 OMT (Calypte Biomedical Corp.), Capillus™ HIV-1/HIV-2 (Trinity Biotech), Care Start™ HIV 1-2-O (Access Bio, Inc.), Clearview® COMPLETE HIV1/2 (Inverness Medical Innovations, Inc.), Clearview® HIV ½ STAT-PAK® Assay (Inverness Medical Innovations, Inc.), Combaids RS Advantage (Span Diagnostics Ltd.), Determine™ HIV-1/2 (Inverness Medical Innovations, Inc./Abbott Laboratories); Double Check™ HIV 1&2 (Inverness Medical Innovations, Inc./Orgenics, Ltd.), Double Check Gold™ HIV 1&2 (Inverness Medical Innovations, Inc./Orgenics, Ltd.), First Response® HIV 1-2.0 (Premier Medical Corporation), Genie II HIV-1/HIV-2 (Bio-Rad Laboratories), HIV 1/2 Rapid Test Kit (Medinostics Int'l), HIV ½ STAT-PAK® Assay (Chembio Diagnostic Systems, Inc.), HIV1/2 STAT-PAK® Dipstick Assay (Chembio Diagnostic Systems, Inc.), Diagnostic kit for HIV (1+2) Antibody (colloidal gold) (Shanghai Kehua Bioengineering), HIV Say 1&2 Rapid SeroTest™ (Savyvon Diagnostics Ltd), ImmunoComb™ HIV 1 &2 (Inverness Medical Innovations, Inc./Orgenics, Ltd.), InstantCHEK™ HIV 1+2 (EY Laboratories), INSTI™ HIV Antibody (Biolytical Laboratories), Multispot™ HIV-1/HIV-2 (Bio-Rad Laboratories), OraQuick® ADVANCE™ Rapid HIV-1/2 (OraSure Technologies), OraQuick® HIV-1/2 Rapid Antibody Test (OraSure Technologies), RAPID 1-2-3® HEMA EZ™ (Hema Diagnostics Systems), RAPID 1-2-3® HEMA EXPRESS™ (Hema Diagnostics Systems), Reveal® G3 Rapid HIV-1 Antibody Test (MedMira), Signal HIV Rapid Test (Span Diagnostics Ltd.), Uni-Gold™ HIV (Trinity Biotech), and Uni-Gold™ Recombigen® HIV (Trinity Biotech).

A subject identified or diagnosed as having an HIV infection or AIDS may be treated using any of the methods described herein. Successful treatment of an HIV infection or AIDS can be assessed by monitoring one of more of the following: the number or the severity, frequency, or duration of one of more symptoms of an HIV infection or AIDS in the subject, the level of CD4⁺ T-cells in the subject, the total number of T-cells or white blood cells (white blood cell count) in the subject, the ratio of CD4⁺ T-cells to CD8⁺ T-cells in the subject, a decrease in the number or severity of opportunistic bacterial, viral, fungal, or parasitic infections in the subject, and a decrease in the titer of HIV in the subject. Based on the observation or determination of one or more of these factors, a health care professional may adjust the therapeutic treatment regime.

### Methods of Treating HIV Infection or AIDS

Provided herein are methods for treating HIV infection or AIDS in a subject (e.g., decreasing the risk of developing or preventing the development of an HIV infection or AIDS in a subject (e.g., a human subject identified as being at risk of a future exposure to HIV (e.g., through participation in unprotected sexual intercourse, exposure to a blood product(s), or exposure to a needle(s) or a sharp object(s) contaminated with HIV), or a subject identified as being at risk of a previous exposure to HIV (e.g., a subject who has previously participated or currently participates in unprotected sexual intercourse, a subject who has been exposed to a blood product(s), or a subject who has been exposed to or punctured with a needle(s) or a sharp object(s) contaminated with HIV), decreasing the severity, frequency, and/or duration of one or more symptoms of an HIV infection or AIDS) in a subject (e.g., a human), increasing the ratio of CD4⁺ T-cells to CD8⁺ T-cells in a subject (e.g., a human) with an HIV infection or AIDS, increasing the number of CD4⁺ T cells in a subject (e.g., a human) with an HIV infection or AIDS, and/or increasing the total T-cell or white blood cell count in a subject (e.g., a human) with a disease (e.g., an HIV infection or AIDS) (e.g., as compared to a control subject having the same disease but not receiving treatment, the same subject prior to treatment, a control population identified as being at future risk for developing HIV but not receiving treatment, or a control population identified as being at risk of a previous exposure to HIV but not receiving treatment)) that include administering to the subject at least one (e.g., one, two, three, or four) human, chimeric, or humanized antibody, or antigen-binding fragment thereof described herein. Also provided herein are methods for decreasing the number or the severity, frequency, and/or duration of one of more symptoms of HIV infection or AIDS in a subject that include administering to the subject at least one (e.g., one, two, three, or four) human, chimeric, or humanized antibody, or antigen-binding fragment thereof described herein.

Any of the antibodies or antibodies fragments described herein can be used alone or in any combination in any of the methods described herein. In some embodiments, the antibody or antigen-binding fragment administered to the subject can bind competitively with an antibody produced by the hybridoma deposited at the American Type Culture Collection (ATCC) and designated as HB 9579. In some embodiments, the antibody or antigen-binding fragment has K_{D} for binding to human CD11a equal to or less than 2 nM (e.g., equal to or less than 2 nM, equal to or less than 1 nM, equal to or less than 800×10⁻¹⁰ M, equal to or less than 500×10⁻¹⁰ M, or equal to or less than 100×10⁻¹⁰ M). In some embodiments, the antibody or antigen-binding fragment thereof binds to an epitope in CD11a that does not overlap with the ICAM-1 binding site of CD11a (when present in the LFA-1 heterodimer). In some embodiments, the binding of the antibody or antigen-binding fragment to CD11a on the surface of a cell (e.g., a human CD8⁺ T-cell) results in the crosslinking of CD11a (e.g., present in LFA-1). In some embodiments, the binding of the antibody or antigen-binding fragment of CD11a on the surface of a CD8⁺ T-cell (e.g., a human CD8⁺ T-cell) results in the crosslinking of CD11a (e.g., present in LFA-1) on the CD8⁺ T-cell surface. Any of the antibodies, antigen-binding antibody fragments, or pharmaceutical compositions described herein can be administered to the subject in any combination.

In some non-limiting embodiments, the antigen-binding fragment can be a Fab fragment, a F(ab′)₂ fragment, or a scFv fragment. In some embodiments, the antibody or antigen-binding fragment thereof can contain a light chain variable domain containing a contiguous sequence of at least 5 amino acids (e.g., at least 5, 10, 15, 20, 25, or 30 amino acids) within the sequence of SEQ ID NO: 1, SEQ ID NO: 19, or SEQ ID NO: 20, and/or a heavy chain variable domain containing a contiguous sequence of at least 5 (e.g., at least 5, 10, 15, 20, 25, or 30 amino acids) within the sequence of SEQ ID NO: 2, SEQ ID NO: 21, or SEQ ID NO: 22. In some embodiments, the antibody or antigen-binding fragment thereof contains a light chain variable domain containing the sequence of SEQ ID NO: 1, and/or a heavy chain variable domain containing the sequence of SEQ ID NO: 2. In some embodiments, the antibody or antigen-binding fragment thereof contains a light chain containing the sequence of SEQ ID NO: 3, and/or a heavy chain containing the sequence of SEQ ID NO: 4. In some embodiments, the antibody or antigen-binding fragment thereof contains a light chain containing the sequence of SEQ ID NO: 20, and/or a heavy chain containing the sequence of SEQ ID NO: 21 or SEQ ID NO: 22. In some embodiments, the antibody or antigen-binding fragment thereof contains a light chain containing the sequence of SEQ ID NO: 20 and a heavy chain containing the sequence of SEQ ID NO: 22. In some embodiments, the antibody is any of the chimeric antibodies described in the examples. In some embodiments, the antigen-binding fragment is derived from any of the chimeric antibodies described in the examples.

In some non-limiting embodiments, the antibody or antigen-binding fragment thereof contains a heavy chain variable domain sequence containing one, two, or three complementary determining regions (CDRs) selected from the group of SFGMH (SEQ ID NO: 13), YISSGSSTLHYA DTVKG (SEQ ID NO: 14), and GSRNLSHRLLS (SEQ ID NO: 15), and/or a light chain variable domain sequence containing one, two, or three CDRs selected from the group of KASQDVSTAVA (SEQ ID NO: 16), WASTRHT (SEQ ID NO: 17), and QQHYTTPWT (SEQ ID NO: 18). In some embodiments, the antibody or antigen-binding fragment thereof contains a heavy chain variable domain sequence containing the complementary determining regions (CDRs) of SFGMH (SEQ ID NO: 13), YISSGSSTLHYA DTVKG (SEQ ID NO: 14), and GSRNLSHRLLS (SEQ ID NO: 15), and/or a light chain variable domain sequence containing the CDRs of KASQDVSTAVA (SEQ ID NO: 16), WASTRHT (SEQ ID NO: 17), and QQHYTTPWT (SEQ ID NO: 18)

In some embodiments of any of the methods described herein, the subject can be administered at least one antibody or antigen-binding fragment thereof that contains at least one CDR within the light chain variable domain sequence of the antibody produced from the hybridoma deposited with the ATCC and designated HB 9579, and/or at least one CDR within the heavy chain variable domain sequence of the antibody produced from the hybridoma deposited with the ATCC and designated HB 9579. In some embodiments of any of the methods described herein, the subject can be administered at least one antibody or antigen-binding fragment thereof that contains the three CDRs within the light chain variable domain sequence of the antibody produced from the hybridoma deposited with the ATCC and designated HB 9579, and/or the three CDRs within the heavy chain variable domain sequence of the antibody produced from the hybridoma deposited with the ATCC and designated HB 9579.

In some embodiments of any of the methods described herein, the at least one antibody can be a human antibody or an antigen-binding fragment thereof, a humanized antibody or an antigen-binding fragment thereof, or a chimeric antibody or an antigen-binding fragment thereof (e.g., generated using any of the methods described herein or known in the art). In some embodiments of any of the methods described herein, the at least one antigen-binding fragment thereof can be a Fab fragment, a F(ab′)2 fragment, or a scFv fragment.

In some embodiments, the antibody or antigen-binding fragment thereof does not induce detrimental immunosuppression in a mammal administered the antibody or antigen-binding fragment thereof. In some embodiments, the antibody or antigen-binding fragment thereof further contains a stabilizing moiety (e.g., a protein, a carbohydrate, or a polymer). In some embodiments, the antibody or antigen-binding fragment thereof is a single chain antibody or a multi-specific (e.g., bi-specific) antibody.

In some embodiments, the human has been diagnosed as having an HIV infection. In some embodiments, the subject has been diagnosed as having AIDS. A subject can be diagnosed as having an HIV infection or AIDS by a medical professional using any of the methods described herein (e.g., by the observation of at least one symptom of an HIV infection or AIDS in a subject) or any methods known in the art.

The administering of a therapeutic agent to a subject identified as being at risk of a future exposure to HIV is known as pre-exposure prophylaxis or PREP. Provided herein are pre-exposure prophylaxis methods of treatment that include administering a therapeutically effective amount of at least one (e.g., one, two, three, or four) human, chimeric, or humanized antibody, or antigen-binding fragment thereof described herein to a subject identified as being at risk of a future exposure to HIV. Also provided herein are pre-exposure prophylaxis methods of treating an HIV infection or AIDS in a subject (e.g., reducing the risk of developing or preventing the development of an HIV infection or AIDS) that include identifying a subject as being at risk of a future exposure to HIV (e.g., through current or future participation in unprotected sexual intercourse, exposure to a blood product(s), or exposure to a needle(s) or a sharp object(s) contaminated with HIV), and administering to a subject identified as being at risk of a future exposure to HIV a therapeutically effective amount of at least one (e.g., one, two, three, or four) human, chimeric, or humanized antibody, or antigen-binding fragment thereof described herein. In some embodiments, at least two (e.g., three, four, or five) doses of at least one human, chimeric, or humanized antibody are administered to the subject.

The administering of a therapeutic agent to a subject identified as being at risk of a previous exposure to HIV is known as post-exposure prophylaxis or PEP. Provided herein are post-exposure prophylaxis methods of treatment that include administering a therapeutically effective amount of at least one (e.g., one, two, three, or four) human, chimeric, or humanized antibody, or antigen-binding fragment thereof described herein to a subject identified as being at risk of a previous exposure to HIV. Also provided herein are post-exposure prophylaxis methods of treating an HIV infection or AIDS (e.g., reducing the risk of developing or preventing the development of an HIV infection or AIDS) in a subject that include identifying a subject as being at risk of a previous exposure to HIV (e.g., through previous or ongoing participation in unprotected sexual intercourse, previous exposure to a blood product(s), or previous exposure to or puncture with a needle(s) or a sharp object(s) contaminated with HIV), and administering to a subject identified as being at risk of a previous exposure to HIV a therapeutically effective amount of at least one (e.g., one, two, three, or four) human, chimeric, or humanized antibody, or antigen-binding fragment thereof described herein. In some embodiments, at least two (e.g., three, four, or five) doses of at least one human, chimeric, or humanized antibody are administered to the subject.

In some embodiments, the subject may be hospitalized or living in an assisted care facility (e.g., a nursing home). The at least one antibody, antigen-binding fragment thereof, or pharmaceutical composition described herein can be self-adminsitered by the subject or can be administered to the subject by a health care professional. In non-limiting examples, the administering can be performed at a health care facility (e.g., a hospital, an assisted living facility, or a clinic).

In some embodiments of any of the methods described herein, the subject is administered at least one (e.g., at least 1, 2, 3, 4, 5, 6, 7, 8, 9, 10, 15, 20, 25, or 30) dose of a composition containing at least one (e.g., one, two, three, or four) of any of the antibodies, antigen-binding fragments, or pharmaceutical compositions described herein. In any of the methods described herein, at least one antibody, antigen-binding fragment, or pharmaceutical composition (e.g., any of the antibodies, antigen-binding fragments, or pharmaceutical compositions described herein) can be administered intravenously, intaarterially, subcutaneously, intraperitoneally, or intramuscularly to the subject. In some embodiments, at least one antibody, antigen-binding fragment, or pharmaceutical composition is administered intravenously.

In some embodiments, the subject is administered at least one antibody, antigen-binding fragment, or pharmaceutical composition (e.g., any of the antibodies, antigen-binding fragments, or pharmaceutical compositions described herein) and at least one additional therapeutic agent. Non-limiting examples of additional therapeutic agents that can be administered to the subject include: Combovir, azidothymidine (AZT), Emtriva, Epivir, Epzicom, Retrovir, Trizivir, Truvada, Videx, Viread, Zerit, Ziagen, Racivir, Amdoxovir, Apricitabine, Elvucitabine, Edurant, Intelence, Rescriptor, Sustiva, Viramune, GSK-2248761, Lersivirine, Agenerase, Aptivus, Crixivan, Invirase, Kaletra, Lexiva, Norvir, Prezista, Reyataz, Viracept, Fuzeon, Selzentry, Vicriviroc, Ibalizumab, PRO 140, Isentess, GSK-572, Elvitegravir, Bevirimat, and Droxia. Further examples of additional therapeutic agents that can be administered to the subject include analgesics (e.g., paracetamol, non-steroidal anti-inflammatory drugs (e.g., aspirin, ibuprofen, and naproxen), codeine, tramadol, DF118 forte, dextropropoxyphene, morphine, methadone, fentanyl, and buprenorphine). In some embodiments, at least one additional therapeutic agent and at least one antibody or antigen-binding fragment thereof (e.g., any of the antibodies or antibody fragments described herein) are administered in the same composition (e.g., the same pharmaceutical composition). In some embodiments, the at least one additional therapeutic agent and the at least one antibody or antigen-binding fragment thereof are administered to the subject using different routes of administration (e.g., at least one additional therapeutic agent delivered by oral administration and at least one antibody or antigen-binding fragment thereof delivered by intravenous administration).

Any of the methods provided herein can achieve one or more of the following in a subject having an HIV infection or AIDS: an increase the CD4⁺ T-cell level, an increase the ratio of CD4⁺ T-cells to CD8⁺ T-cells, and an increase the total number of T-cells or the total white blood cell count (relative to a subject having HIV infection or AIDS not receiving treatment or the same subject prior to treatment). In some embodiments, the methods provided herein result in a decrease in the number or a decrease in the severity, frequency, or duration of at least one symptom of an HIV infection or AIDS (e.g., any of the symptoms described herein) (relative to a subject having HIV infection or AIDS not receiving treatment or the same subject prior to treatment). Non-limiting examples of symptoms of an HIV infection or AIDS include: headache, sore throat, swollen lymph glands, mild infections, diarrhea, weight loss, cough, shortness of breath, soaking night sweats, shaking chills or fever, persistent white spots or unusual lesions on the tongue or in mouth, persistent unexplained fatigue, blurred or distorted vision, and skin rashes or bumps.

In some embodiments, the administering of at least one antibody or antigen-binding fragment thereof reduces an opportunistic infection in a subject having an HIV infection or AIDS or reduces the risk of developing an opportunistic infection in a subject having an HIV infection or AIDS. For example, the methods described herein may result in a significant decrease in the number or the severity, frequency, or duration of one or more symptoms of an opportunistic bacterial, fungal, viral, or parasitic infection (e.g., any of the exemplary opportunistic bacterial, fungal, viral, or parasitic infections described herein) in a subject having an HIV infection or AIDS that has such an opportunistic infection. In some embodiments, the methods decrease the risk of developing an opportunistic bacterial, viral, fungal, or parasitic infection (e.g., any of the exemplary opportunistic bacterial, fungal, viral, or parasitic infections described herein) in a subject having an HIV infection or AIDS and not having an opportunistic bacterial, viral, fungal, or parasitic infection (e.g., as compared to a subject or group of subjects having an HIV infection or AIDS and not receiving the treatment or receiving an alternative form of treatment). In some embodiments, the methods described herein decrease the risk of developing cancer (e.g., any of the exemplary cancers described herein) in a subject having an HIV infection or AIDS (e.g., as compared to a subject or group of subjects having an HIV infection or AIDS and not receiving the treatment or receiving an alternative form of treatment). In some embodiments, the administering of at least one antibody or antigen-binding fragment thereof, or a composition containing at least one antibody or antigen-binding thereof results in a decrease (e.g., a significant or detectable decrease, e.g., at least a 5%, 10%, 15%, 20%, 25%, or 30% decrease) in HIV titer in the subject.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents can be administered to the subject after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of HIV infection or AIDS in a subject or HIV titers in the subject). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art). A skilled medical professional can further determine when to discontinue treatment (e.g., for example, when the subject's CD4⁺ T-cell levels reach a level present in uninfected or control subjects).

In some embodiments, the administration of at least one antibody, antigen-binding antibody fragment, or pharmaceutical composition described herein does not cause detrimental immunosuppression in the subject. Determintal immunosuppression, for example, can be indicated by a nearly complete suppression of CD8⁺ T-cell activation (cytotoxic T-cell activation) in a subject following therapeutic treatment.

### Methods of Crosslinking CD11a

Also provided herein are methods of crosslinking CD11a (e.g., present in LFA-1) on the surface of a cell (e.g., a CD8⁺ T-cell) that include contacting a cell expressing CD11a in the plasma membrane with at least one antibody, antigen-binding antibody fragment, or pharmaceutical composition (e.g., at least one of the antibodies, antigen-binding antibody fragments, or pharmaceutical compositions described herein) that mediates crosslinking of CD11a on the surface of the cell. In some embodiments, the CD11a that is crosslinked is present in LFA-1 (a heterodimer of CD11a and CD18). In some embodiments, the cell (e.g., a CD8⁺ T-cell, such as a human CD8⁺ T-cell) is in vitro. In some embodiments, the cell (e.g., CD8⁺ T-cell) is in vivo (e.g., in a human).

The at least one antibody or antigen-binding antibody fragment can be formulated or administered using any of the methods described herein. In some embodiments, the subject has a retroviral infection (e.g., an HIV infection) or AIDS. In some embodiments, the subject has been diagnosed as having a retroviral infection (e.g., an HIV infection) or AIDS prior to the administration of the at least one antibody, antigen-binding antibody fragment, or pharmaceutical composition.

### EXAMPLES

The invention is further described in the following examples, which do not limit the scope of the invention described in the claims.

### Example 1

### Production of a Chimeric Anti-CD11a Antibody

The S6F 1 antibody is a mouse antibody that was described in U.S. Pat. No. 5,002,869. U.S. Pat. No. 6,534,057 describes that administration of the mouse S6F1 antibody to human subjects having an HIV infection resulted in an increase in the level of CD4+ T-cells and an increase in the ratio of CD4+ T-cells to CD8+ T-cells, and thus did not result in detrimental immunosuppression in human subjects. Described herein is the specific epitope on CD11a that is recognized by the mouse S6F1 antibody that can be used as the target for the treatment of an HIV infection or AIDS. Also provided herein are human, humanized, and chimeric antibodies that specifically bind to this particular epitope on CD11a.

### Generation of a Chimeric Light Chain Immunoglobulin

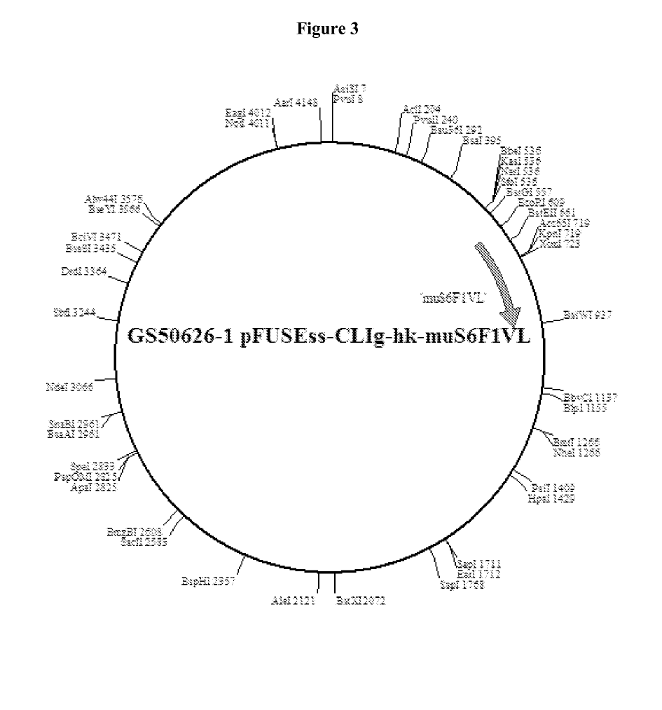

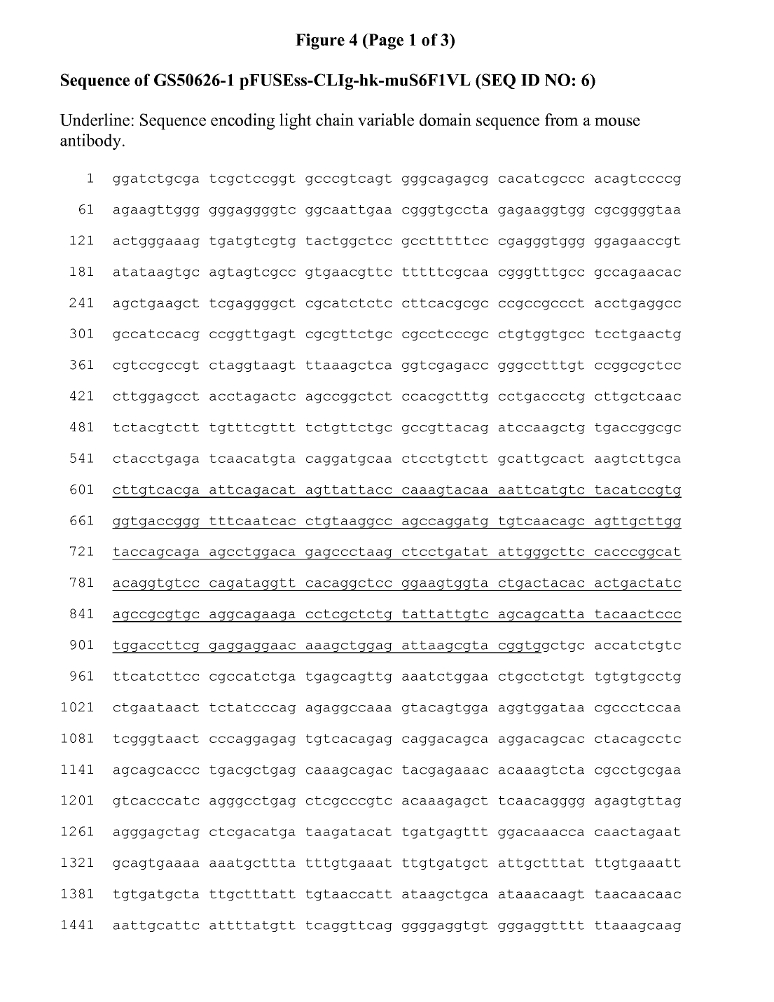

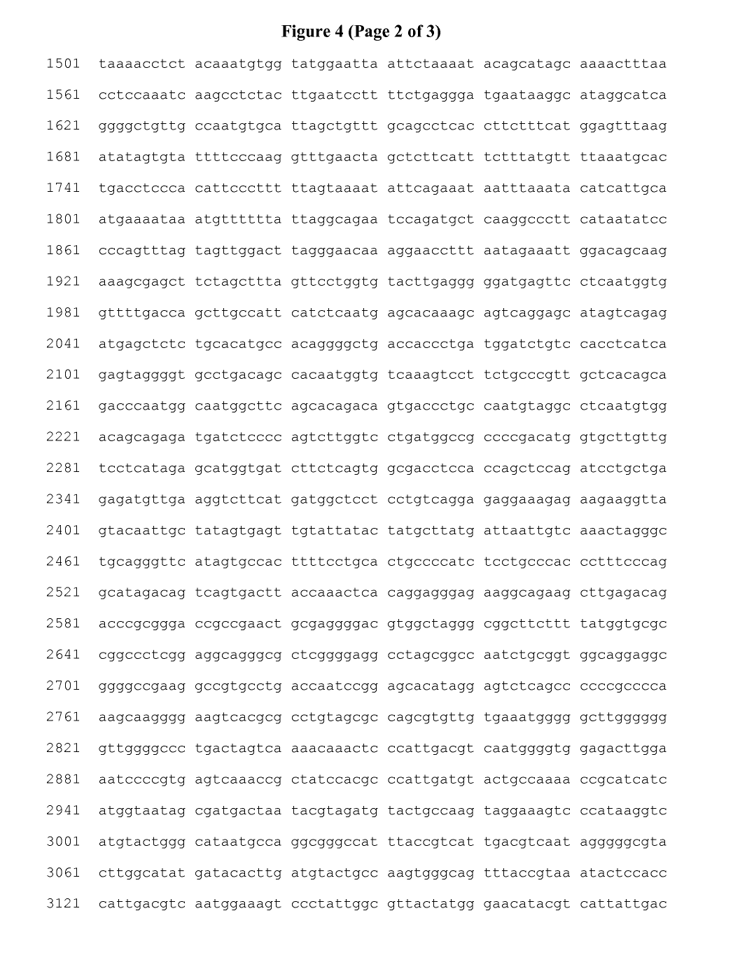

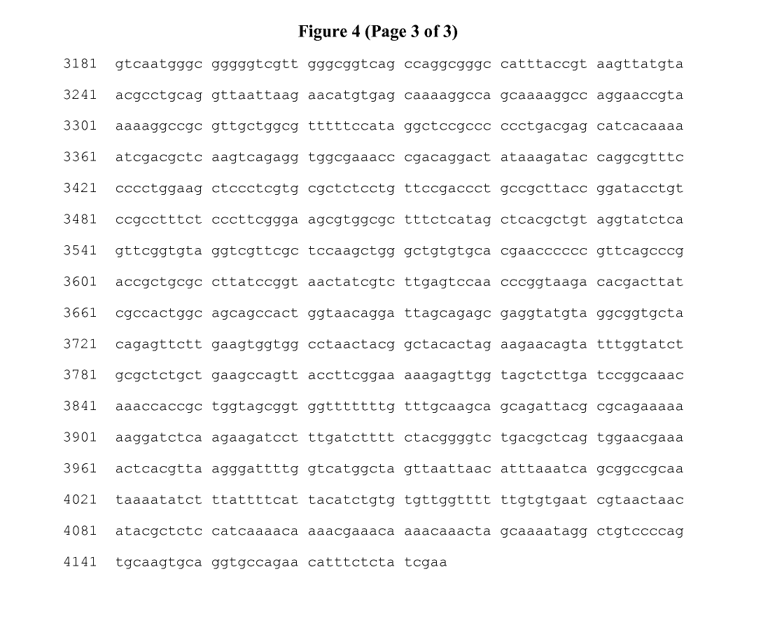

The DNA sequence encoding the light chain variable domain of the antibody produced from the hybridoma deposited with the ATCC and designated HB 9579 (the S6F1 antibody) was sequenced and optimized for codon usage in Chinese hamster ovary (CHO) cells and human embryonic kidney (HEK) 3 cells. The resulting optimized sequence encoding the light chain variable domain sequence of the S6F 1 antibody is underlined in FIG. 4 (nucleotides 601-945 of SEQ ID NO: 6). This optimized sequence was inserted into the pFUSE2ss-CLIg-hk vector (Invivo Gen) to generate the GS50626-1 pFUSEss-CLIg-hk-muS6F 1VL vector (FIG. 3). The pFUSE2ss-CLIg-hk parent vector contains a sequence encoding the interleukin-2 (IL2) leader sequence and the human constant (Fc) domain for the human κ immunoglobulin light chain. The optimized sequence encoding the light chain variable domain sequence of the S6F1 antibody is inserted immediately 3′ of the IL2 leader sequence, and immediately 5′ of the sequence encoding the human constant domain for the human κ immunoglobulin light chain (both of these sequences are indicated as “muS6F1VL” in the plasmid diagram shown in FIG. 3). The complete sequence of the resulting pFUSE2ss-CLIg-hk vector is shown in FIG. 4 (SEQ ID NO: 6).

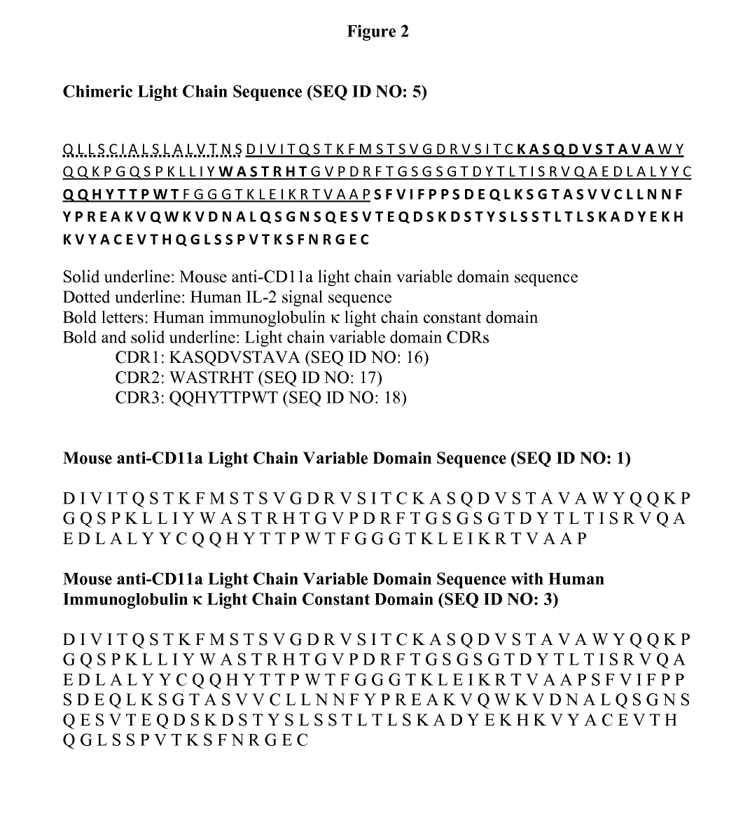

The resulting GS50626-1 pFUSE2ss-CLIg-hk-muS6F1VL vector encodes a light chain immunoglobulin that contains from the N- to C-terminus: the IL-2 signal sequence (amino acids 1-16 of SEQ ID NO: 5), the S6F1 light chain variable domain sequence (SEQ ID NO: 1 or amino acids 17-129 of SEQ ID NO: 5), and a human κ immunoglobulin light chain constant domain sequence (amino acids 130-230 of SEQ ID NO: 5) (FIG. 2). Following cellular processing to remove the IL-2 signal sequence, the resulting light chain immunoglobulin protein has the sequence of SEQ ID NO: 3 (FIG. 2).

### Generation of a Chimeric Heavy Chain Immunoglobulin

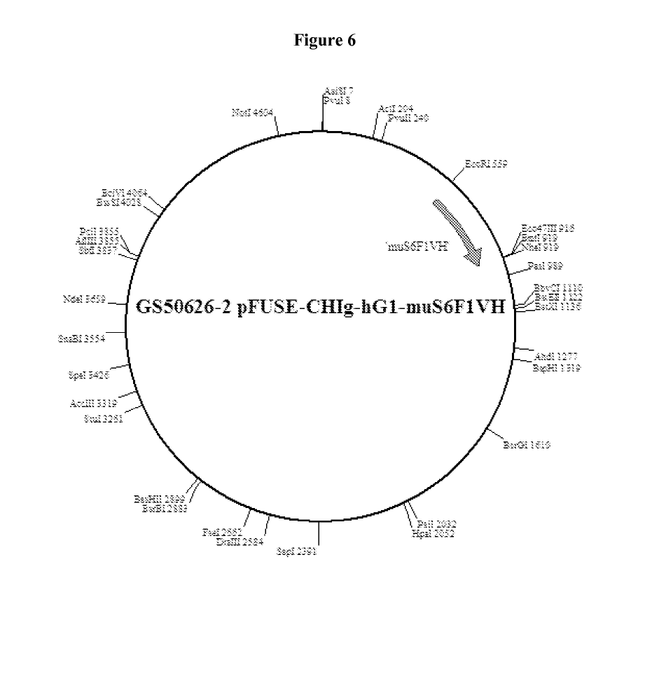

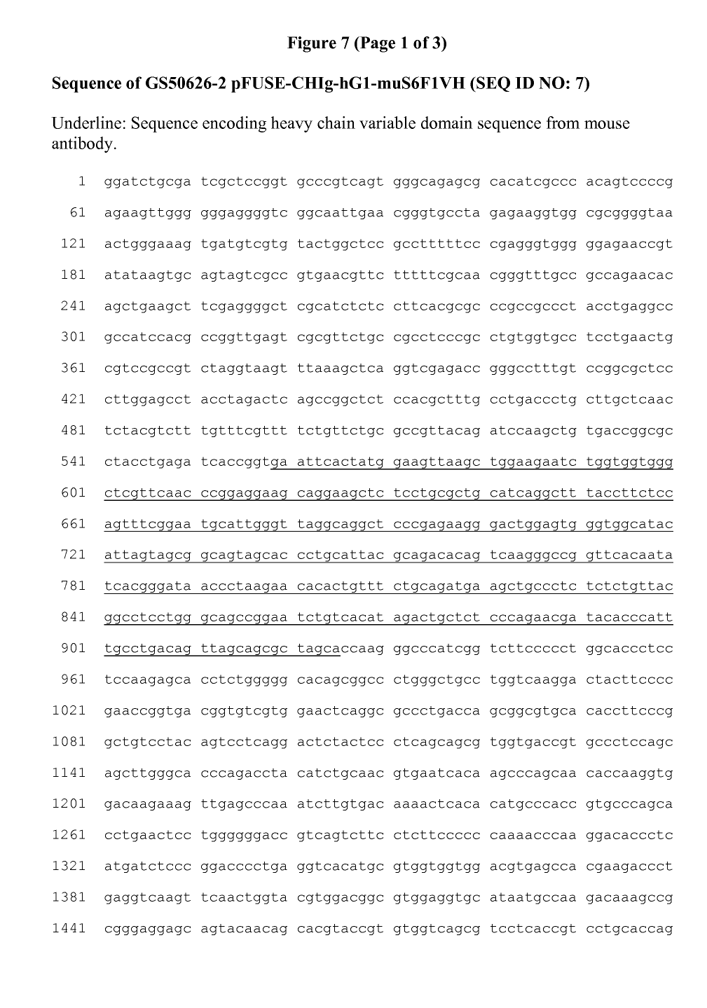

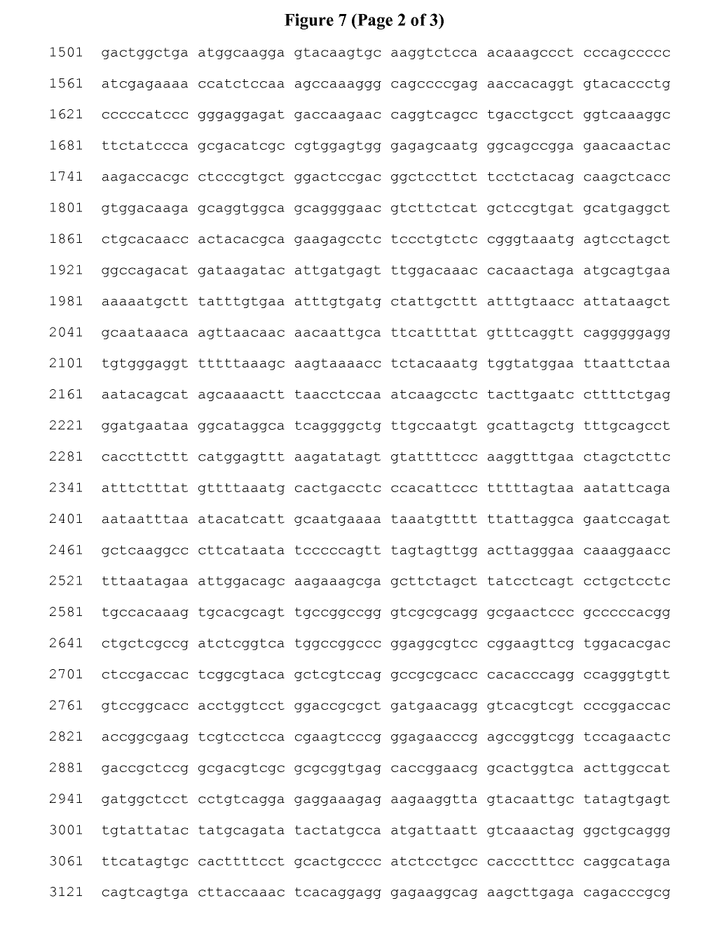

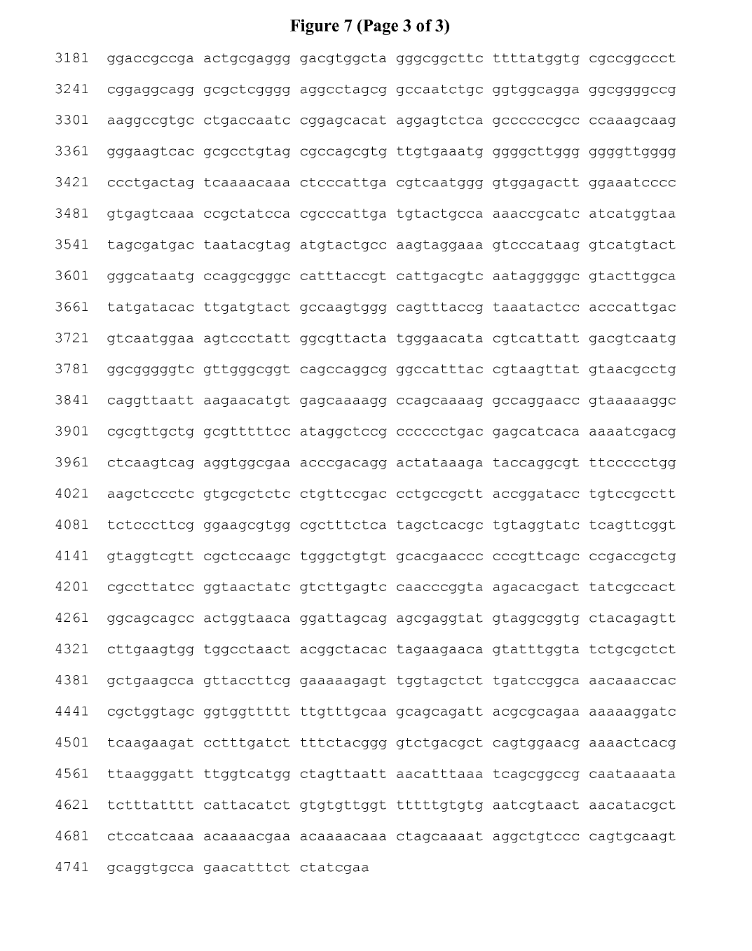

A chimeric heavy chain immunoglobulin was generated using methods similar to those used to generate the chimeric light chain immunoglobulin described above. The DNA sequence encoding the heavy chain variable domain of the antibody produced from the S6F1 antibody was sequenced and optimized for codon usage in CHO cells and HEK3 cells. The resulting optimized sequence encoding the heavy chain variable sequence of the S6F1 antibody is underlined in FIG. 7 (nucleotides 559-925 of SEQ ID NO: 7). This optimized sequence was inserted into the pFUSE-CHIg-hG1-VH vector (Invivo Gen) to generate the GS50626-2 pFUSEss-CLIg-hG1-muS6F1VH vector (FIG. 6). The pFUSE-CHIg-hG1-VH parent vector contains a sequence encoding the human constant (Fc) domain of the human IgG heavy chain. The optimized sequence encoding the heavy chain variable domain sequence of the S6F1 antibody is inserted immediately 5′ of the sequence encoding the constant domain of the human IgG heavy chain (both of these sequences are indicated as “muS6F1VH” in the plasmid diagram shown in FIG. 6). The complete sequence of the resulting GS50626-2 pFUSE-CHIg-hG1-VH-muS6F1VH vector is shown in FIG. 7 (SEQ ID NO: 7).

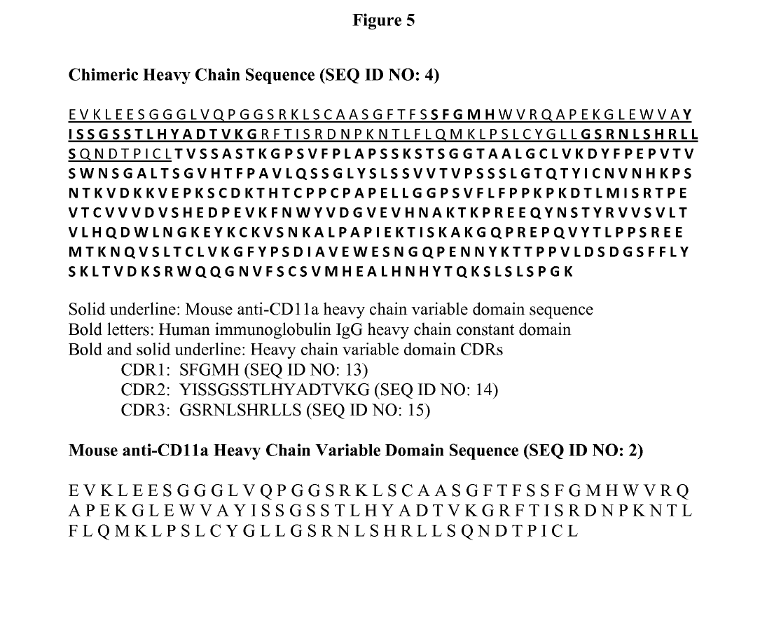

The resulting GS50626-2 pFUSE2ss-CLIg-VH-muS6F1VH vector encodes a heavy chain immunoglobulin that contains from the N- to C-terminus: the S6F1 heavy chain variable domain sequence (SEQ ID NO: 2 or amino acids 1-112 of SEQ ID NO: 4; FIG. 5), and the constant domain of the human IgG heavy chain (amino acids 113-446 of SEQ ID NO: 4; FIG. 5). The resulting chimeric heavy chain sequence has the sequence of SEQ ID NO: 4 (FIG. 5).

The resulting GS50626-1 pFUSE2ss-CLIg-hk-muS6F1VL or the GS50626-2 pFUSE2ss-CLIg-VH-muS6F1VH vector described above were used to transfect E. coli. As the GS50626-1 pFUSE2ss-CLIg-hk-muS6F1VL vector contained a blastidicin-resistance gene and the GS50626-2 pFUSE2ss-CLIg-VH-muS6F1VH vector contained a zeocin-resistance gene, colonies resistant to blastidicin or zeocin (containing the GS50626-1 pFUSE2ss-CLIg-hk-muS6F1VL or GS50626-2 pFUSE2ss-CLIg-hk-muS6F1VH vector) were used to purify the plasmids. The resulting plasmids were sequenced to confirm the nucleotide sequence encoding the chimeric light or heavy chain immunoglobulin.

The chimeric antibodies expressed from the GS50626-1 pFUSE2ss-CLIg-hk-muS6F1VL and the GS50626-2 pFUSE2ss-CLIg-VH-muS6F1VH vectors described above were produced in A293 cells. Briefly, A293 cells were transfected with the GS50626-1 pFUSE2ss-CLIg-VH-muS6F1VL vector and the GS50626-2 pFUSE2ss-CLIg-hk-muS6F 1VH vector at a ratio of 1 to 2 under the conditions for transient transfection, and the cells were cultivated for an additional 5 days. The transfection methods and the protocol for purifying the chimeric antibodies are described in detail below.

### Transfection Protocol

The cells used to express the recombinant chimeric antibody were A293-2 cells from Millipore. The cells used had a viability greater than 93%, and a passage number greater than 3 and less than 25. The cells were passaged twice weekly in Hyclone SFM4HEK 293 media (Cat#SH30521.01) by reducing the viable cell concentration to 0.3×10⁶ cells/mL. The cells were transfected using Targefact-293FS reagent (Targeting Systems; Cat#293F-01) in Life Technologies OPTI-MEM Reduced Serum Medium (Cat#31985-088). This reagent was stored at −20° C., and thawed to room temperature immediately prior to use. The reagent was vortexed at full speed for 30 seconds, 2-3 times, immediately prior to use.

A culture of A293-S cells at a density of 2 million cells/mL (in 50 mL or more) was prepared for transient transfection using the method described below.

(A) Two 125-mL Corning shaker flasks were obtained. One was used to grow control cells, and the other was used to culture the transfected cells that express the chimeric antibody.

(B) Cells and media (Hyclone SFM4HEK 293) were added to each flask to achieve a total volume of 27 mL, and a cell density of 1.1×10⁶ cells/mL. Ten μL of a 1.7 mg/mL solution of aprotinin was added per 10 mL of culture medium.

(C) The flasks were placed in a shaker incubator (125 rpm and 5% CO₂).

(D) Both the DNA and Targefect-293FS Reagent were thawed on ice. The Targefect-293FS Reagent was vortexed vigorously for 30-60 seconds before use.

(E) Two 15-mL conical tubes were prepared by adding 750 mL of OPTI-MEM media (Invitrogen) to each tube. One tube was labeled “DNA tube A,” and the other was labeled “DNA tube B.”

(F) Twenty μg of light chain DNA was added to “DNA tube A,” and the solution was mixed by flicking the bottom of the tube six times.

(G) Ten μg of heavy chain DNA was added to “DNA tube B,” and the solution was mixed by flicking the bottom of the tube six times.

(H) Two 15-mL conical tubes were prepared by adding 750 μL of OPTI-MEM media (Invitrogen) to each tube. One tube was labeled “Lipid tube A,” and the other was labeled “Lipid tube B.”

(I) Fifteen μL of Targefect-293FS was added to “Lipid tube A,” and mixed by flicking the bottom of the tube six times.

(J) Fifteen μL of Targefect-293FS was added to “Lipid tube B,” and mixed by flicking the bottom of the tube six times.

(K) The DNA and lipid mixtures were incubated for 5 minutes at room temperature.

(L) At the end of the 5 minute incubation period, the DNA mixture was added to the lipid mixture, and the combined solution was mixed by flicking the bottom of the tube six times. In this step, the contents of DNA tube A and Lipid tube A were mixed, and the contents of DNA tube B and Lipid tube B were mixed.

(M) The DNA-lipid mixtures were incubated for 20 minutes at room temperature.

(N) At the end of the 20 minute incubation, 765 μL of each DNA-lipid mixture (the “A” mixture, then the “B” mixture) was added to one of the two 125 mL shaker flasks of cells (labeled as “Antibody Production/Expression”). Three mL of OPTI-MEM was added to the other 125 mL shaker flask of cells (labeled as “Control”). The final cell density of each flask was 1×10⁶ cells/mL.

(O) Both flasks were harvested after 5 days of incubation. The cells were counted in each flask, and the percent cell viability, viable cell counts, and total (live plus dead) cell counts were recorded. The cells were centrifuged at 2,200 rpm, and the supernatant was saved. Aliquots of the supernatant were stored at −80° C.

Chimeric antibodies that bind to substantially the same epitope as the S6F1 antibody can be purified from the cell culture supernatant using methods known in the art (e.g., protein A or protein G purification). For example, the antibodies can be eluted from a protein A resin using 0.5 M arginine buffer (pH 4.3).

### Example 2

### Surface Plasmon Resonance to Determine the Affinity of the Chimeric Antibody

The CD11a binding affinity of the chimeric antibody described in Example 1 was determined using surface plasmon resonance (SPR). These experiments were performed using an ICX SensiQ instrument. In these experiments, Protein G on the SPR surface was used to capture the chimeric anti-CD11a antibody, and then the test antibody was exposed to the extracellular domain of CD11a (SEQ ID NO: 11).

Protein G was covalently attached to the SPR surface using the following reagents: N-(3-Dimethylaminopropyl)-N′-ethylcarbodiimide hydrochloride (EDC), N-hydroxysuccinimide (NHS), acetate buffer (0.01 M, pH 4.5), glacial acetic acid, sodium acetate, protein G, and ethanolamine. The following solutions were prepared and used, as described below, to covalently attach Protein G to the SPR surface.

Solution A (0.4 M EDC solution): 767 mg of EDC was added to 10 mL of deionized water. The solution was filtered through a syringe filter (<1 μm MW cut-off). Fifty μL aliquots of the solution were placed in vials and stored at −20° C. for up to two months.

Solution B (0.1 M NHS): 115 mg of NHS was added to 10 mL of deionized water. Fifty μL aliquots of the solution were placed in vials and stored at −20° C. for up to two months.

Solution C (1 M ethanolamine): 14.64 grams of ethanolamine hydrochloride were added to a container and dissolved in 140 mL of deionized water. The pH of the solution was adjusted to pH 8.0 using 1 M NaOH. The solution was then diluted with deionized water to a final volume of 150 mL.

Solution D (acetate buffer): 80 mg of sodium acetate was added to 100 mL of deionized water. The pH of the solution was adjusted to pH 4.5 by the addition of approximately 375 μL of glacial acetic acid. Fine adjustments in the pH were made by adding small volumes of acetate or acetic acid to the solution (as needed). The solution was then filtered and stored at 4° C. for up to 3 months.

Solution E (HEPES buffered saline): HEPES buffered saline (HBS) was prepared using methods known in the art. HBS is prepared by dissolving 8.766 g of NaCl and 5.2 g of HEPES in 900 mL of water, adjusting the pH to 7.0, and bringing the total volume of the solution to 1 L using water. The HBS should be filtered and degassed using a cellular acetate filter. The prepared HBS is used as a running buffer in the SensiQ system. The SensiQ system should be primed using HBS before performing the SPR experiments.

Immobilization Procedure: A 0.1 M HCl solution (100 μL at 50 μL/minute) was used to clean the chip surface before use. The loop was then purged with 2.5 mL deionized water. The baseline was stabilized for 5 minutes before beginning the immobilization procedure.

A 200 μL solution of NeutrAvidin (100 μg/mL) was prepared in 10 mM acetate buffer (pH 4.5). No free amines were present in this solution, in the running buffer, or in the fluidics system. The sample loading syringes were rinsed in deionized water and blown out with air, prior to use.

Pre-concentration Test: A pre-concentration test was performed prior to the coupling of protein G to the surface of the chip. The steps of this step are listed below.

(A) The flow rate was set to 25 μL/minute.

(B) The loop was purged with 2.5 mL of deionized water.

(C) Sixty μL of protein solution was loaded into the loop, and 50 μL was injected at a flow rate of 25 μL/minute.

(D) Fifty μL of 10 mM HCl at a flow rate of 50 μL/minute was injected to remove any weakly absorbed protein.

(E) The loop was purged with 2.5 mL of deionized water.

Ligand Coupling After the pre-concentration efficiency of the protein G sample was confirmed, covalent immobilization of protein G on the surface of the chip was performed. In this method, the surface of the chip was activated by converting a small fraction (<20%) of the surface carbonyl groups to amine-reactive N-hydroxysuccinimide esters. The steps of this procedure are listed below.

(A) The flow rate was set to 10 μL/minute.

(B) A vial of the EDC solution and the NHS solution (described above) were thawed and mixed together in a 1:1 ratio. Ten μL of the mixture was added to 990 μL of filtered and degassed deionized water. One hundred μL of this activation solution was injected at a flow rate of 50 μL/minute.

(C) One hundred sixty μL of protein G solution was loaded into the sample loop, and 150 μL of the protein solution was injected at a flow rate of 10 μL/minute.

(D) One hundred μL of 1 M ethanolamine (pH 8.0) was co-injected at a flow rate of 25 μL/minute.

Affinity Assay: An affinity assay was performed after the protein G was coupled to the surface of the chip. In this assay, degassed 10 mM HEPES, 150 mM NaCl, 3.4 mM EDTA, 0.0005% Tween 20 (pH 7.4) was used as the running buffer. A blank was typically run to help compensate for any baseline shift, and Qdat software was used to analyze the generated response curves.

(A) Fifty μL of the chimeric antibody (concentration adjusted to see a response of about 150 to 200 RU) was injected at a rate of 25 μL/minute (dissociation 120 seconds).

(B) Fifty μL of the recombinant extracellular domain of CD11a (50 nM) was co-injected at a rate of 25 μL/minute (dissociation 600 seconds). The extracellular domain of CD11a (SEQ ID NO: 11) was cloned and expressed in E. coli using a histidine tag (described herein). The recombinant extracellular domain of CD11a was purified using a metal chelate affinity column using methods known in the art.

(C) The surface was regenerated by coinjection of 25 μL of 20 mM NaOH at a flow rate of 25 μL/minute (dissociation 0 seconds).

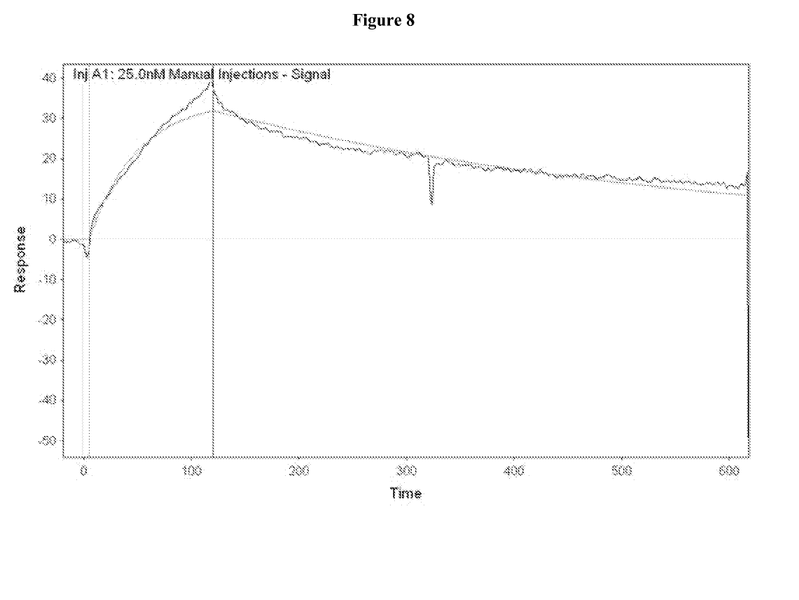

Using these methods, the affinity of a chimeric antibody having a light chain of SEQ ID NO: 3 and a heavy chain of SEQ ID NO: 4 for the extracellular domain of CD11a (SEQ ID NO: 11) was determined to be 1-2 nM (FIG. 8).

### Example 3

### Additional Chimeric Antibodies

Two additional chimeric light chain immunoglobulin molecules and two additional chimeric heavy chain immunoglobulin molecules were generated that have the ability to specifically bind to substantially the same epitope recognized by the antibody produced by the hybridoma deposited at the ATCC and designated as HB 9579. These chimeric immunoglobulin molecules were generated using the methods generally described in Example 1.

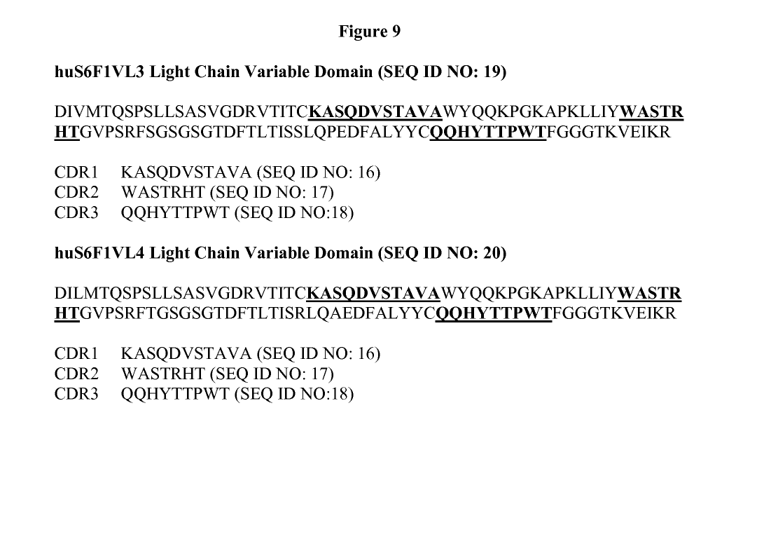

The light chain variable domain for the two additional chimeric light chain immunoglobulin molecules are shown in FIG. 9 (SEQ ID NOS: 19 and 20). The CDRs within the light chain variable domain of each light chain immunoglobulin molecule are also shown in FIG. 9 (bold and underlined; SEQ ID NOS: 16-18).

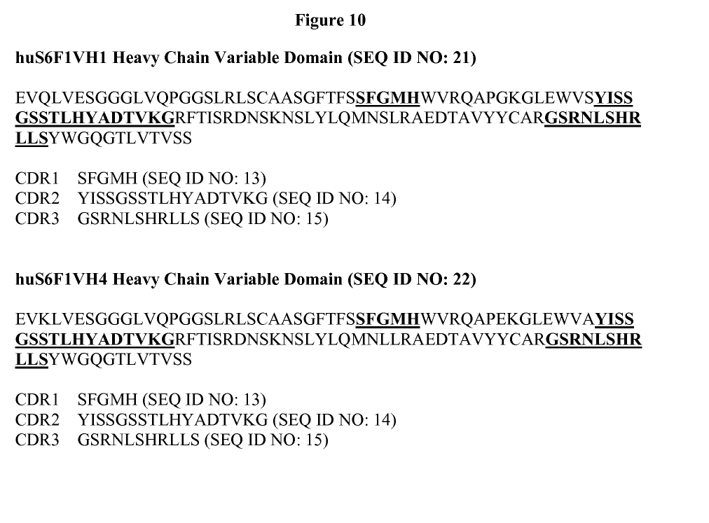

The heavy chain variable domain for the two additional chimeric heavy chain immunoglobulin molecules are shown in FIG. 10 (SEQ ID NOS: 21 and 22). The CDRs within the heavy chain variable domain of each heavy chain immunoglobulin molecule are also shown in FIG. 10 (bold and underlined; SEQ ID NOS: 13-15). The two additional chimeric heavy chain immunoglobulin molecules were able to bind to the recombinant extracellular domain of CD11a (SEQ ID NO: 11) when combined with the light chain of the antibody produced by the hybridoma deposited at the ATCC and designated as HB 9579.

Chimeric light chain immunoglobulin molecules containing SEQ ID NO: 19 or SEQ ID NO: 20 were able to bind to the recombinant extracellular domain of CD11a (SEQ ID NO: 11) when combined with the heavy chain of the antibody produced from the hybridoma deposited at the ATCC and designated as HB 9579. Two chimeric antibodies including a chimeric light chain immunoglobulin containing the sequence of SEQ ID NO: 20 were shown to bind to the recombinant extracellular domain of CD11a (SEQ ID NO: 11) with an affinity similar to that of the antibody produced from the hybridoma deposited at the ATCC and designated as HB9579: a chimeric immunoglobulin light chain containing the sequence of SEQ ID NO: 20 paired with a chimeric immunoglobulin heavy chain containing the sequence of SEQ ID NO: 21 or SEQ ID NO: 22. Of these two chimeric antibodies, the chimeric antibody containing a chimeric light chain containing the sequence of SEQ ID NO: 20 and a chimeric heavy chain containing the sequence of SEQ ID NO: 22 had the strongest affinity for the extracellular domain of CD11a (a fragment of CD11a that contains the unique epitope described herein).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated human, chimeric, or humanized antibody or antigen-binding fragment thereof, wherein the antibody or fragment thereof binds competitively with an antibody produced by the hybridoma deposited at the American Type Culture Collection (ATTC) and designated as HB 9579, and wherein the antibody or antigen-binding fragment thereof comprises a light chain variable domain comprising a contiguous sequence of at least five amino acids within the sequence of SEQ ID NO: 1, SEQ ID NO: 19, or SEQ ID NO: 20, or a heavy chain variable domain comprising a contiguous sequence of at least five amino acids within the sequence of SEQ ID NO: 2, SEQ ID NO: 21, or SEQ ID NO: 22.

2. The antibody or antigen-binding fragment of claim 1, wherein the antibody or antigen-binding fragment thereof has a K_{D} for binding to CD11a equal to or less than 2 nM.

3. The antibody or antigen-binding fragment of claim 1, wherein the antibody or antigen-binding fragment thereof is capable of binding CD11a on the surface of a human cell.

4. The antibody or antigen-binding fragment of claim 3, wherein the antibody or antigen-binding fragment thereof is capable of crosslinking CD11a on the surface of the human cell.

5. The antibody or antigen-binding fragment of claim 3, wherein the human cell is a CD8⁺ T-cell.

6. The antibody or antigen-binding fragment of claim 1, wherein the antigen-binding fragment is selected from the group of: a Fab fragment, a F(ab′)₂ fragment, and a scFv fragment.

7. (canceled)

8. The antibody or antigen-binding fragment of claim 1, wherein the antibody or antigen-binding fragment thereof comprises a light chain variable domain comprising a contiguous sequence of at least five amino acids within the sequence of SEQ ID NO: 1, and a heavy chain variable domain comprising a contiguous sequence of at least five amino acids within the sequence of SEQ ID NO: 2.

9. The antibody or antigen-binding fragment of claim 1, wherein the antibody or antigen-binding fragment thereof comprises a light chain variable domain comprising a contiguous sequence of at least five amino acids within the sequence of SEQ ID NO: 20, and a heavy chain variable domain comprising a contiguous sequence of at least five amino acids within the sequence of SEQ ID NO: 21 or SEQ ID NO: 22.

10. The antibody or antigen-binding fragment of claim 9, wherein the antibody or antigen-binding fragment thereof comprises a contiguous sequence of at least five amino acids within the sequence of SEQ ID NO: 20, and a heavy chain variable domain comprising a contiguous sequence of at least five amino acids within the sequence of SEQ ID NO: 22.

11. The antibody or antigen-binding fragment of claim 1, wherein the antibody or antigen-binding fragment thereof comprises a light chain variable domain comprising the sequence of SEQ ID NO: 1, or a heavy chain variable domain comprising the sequence of SEQ ID NO: 2.

12. The antibody or antigen-binding fragment of claim 9, wherein the antibody or antigen-binding fragment thereof comprises a light chain variable domain comprising the sequence of SEQ ID NO: 20, and a heavy chain variable domain comprising the sequence of SEQ ID NO: 21 or SEQ ID NO: 22.

13. The antibody or antigen-binding fragment of claim 12, wherein the antibody or antigen-binding fragment thereof comprises a light chain variable domain comprising the sequence of SEQ ID NO: 20, and a heavy chain variable domain comprising the sequence of SEQ ID NO: 22.

14. The antibody or antigen-binding fragment of claim 1, wherein the antibody or antigen-binding fragment thereof comprises a heavy chain variable domain sequence containing one, two, or three complementary determining regions (CDRs) selected from the group consisting of SFGMH (SEQ ID NO: 13), YISSGSSTLHYADTVKG (SEQ ID NO: 14), and GSRNLSHRLLS (SEQ ID NO: 15), or a light chain variable domain sequence containing one, two, or three CDRs selected from the group consisting of KASQDVSTAVA (SEQ ID NO: 16), WASTRHT (SEQ ID NO: 17), and QQHYTTPWT (SEQ ID NO: 18).

15. The antibody or antigen-binding fragment of claim 14, wherein the antibody or antigen-binding fragment thereof comprises a heavy chain variable domain sequence containing the CDRs of SFGMH (SEQ ID NO: 13), YISSGSSTLHYADTVKG (SEQ ID NO: 14), and GSRNLSHRLLS (SEQ ID NO: 15), and a light chain variable sequence containing the CDRs of KASQDVSTAVA (SEQ ID NO: 16), WASTRHT (SEQ ID NO: 17), and QQHYTTPWT (SEQ ID NO: 18).

16. The antibody or antigen-binding fragment of claim 15, wherein the antibody or the antigen-binding fragment thereof comprises a light chain variable domain comprising the sequence of SEQ ID NO: 1, and a heavy chain variable domain comprising the sequence of SEQ ID NO: 2.

17. The antibody or antigen-binding fragment of claim 14, wherein the antibody or antigen-binding fragment thereof comprises a light chain comprising the sequence of SEQ ID NO: 3 or a heavy chain comprising the sequence of SEQ ID NO: 4.

18. The antibody or antigen-binding fragment of claim 15, wherein the antibody or antigen-binding fragment thereof comprises a light chain comprising the sequence of SEQ ID NO: 3 and a heavy chain comprising the sequence of SEQ ID NO: 4.

19. The antibody or antigen-binding fragment of claim 14, wherein the antibody or antigen-binding fragment thereof comprises a light chain variable domain comprising the sequence of SEQ ID NO: 19 or SEQ ID NO: 20, or a heavy chain variable domain comprising the sequence of SEQ ID NO: 21 or SEQ ID NO: 22.

20. The antibody or antigen-binding fragment of claim 15, wherein the antibody or antigen-binding fragment thereof comprises a light chain variable domain comprising the sequence of SEQ ID NO: 19 or SEQ ID NO: 20, and a heavy chain variable domain comprising the sequence of SEQ ID NO: 21 or SEQ ID NO: 22.

21. The antibody or antigen-binding fragment of claim 1, wherein the antibody or antigen-binding fragment thereof does not induce detrimental immunosuppression in a mammal administered the antibody or antigen-binding fragment thereof.

22. The antibody or antigen-binding fragment of claim 1, wherein the antibody or antigen-binding fragment thereof is covalently linked to a stabilizing moiety.

23. The antibody or antigen-binding fragment of claim 22, wherein the stabilizing moiety is a protein, a carbohydrate, or a polymer.

24. The antibody or antigen-binding fragment of claim 1, wherein the antibody or antigen-binding fragment thereof is labeled.

25. The antibody or antigen-binding fragment of claim 1, wherein the antibody is a single chain antibody or a multimeric antibody.

26. A pharmaceutical composition comprising the antibody or antigen-binding fragment of claim 1.

27. A method of treating human immunodeficiency virus (HIV) infection in a subject, the method comprising administering a pharmaceutically effective amount of the antibody or antigen-binding fragment thereof of claim 1 to a subject having an HIV infection.

28-40. (canceled)
